(19) United States
(12) Patent Application Publication
(10) Pub. No.: US 2023/0226061 A1
(43) Pub. Date: Jul. 20, 2023

(54) COMBINATION CANCER THERAPY WITH DYRK1 INHIBITORS AND INHIBITORS OF THE RAS-RAF-MEK-ERK (MAPK) PATHWAY

(71) Applicant: Felicitex Therapuetics, Inc., Natick, MA (US)

(72) Inventors: Maria Vilenchik, Newton, MA (US); Michael Frid, Medford, MA (US); Alexandra Kuznetsova, Natick, MA (US); Karina Barseguian, Shrewsbury, MA (US); Marc Duey, Chester Springs, PA (US)

(21) Appl. No.: 17/579,876

(22) Filed: Jan. 20, 2022

(51) Int. Cl. A61K 31/519; A61K 31/475; A61K 45/06; A61K 41/00

(52) U.S. Cl. CPC A61K 31/519; A61K 31/475; A61K 45/06; A61K 41/0038

(57) ABSTRACT

The present invention provides compositions and methods for the treatment of neoplasms, in particular, by targeting of quiescent and proliferating cancers cells with DYRK1 inhibitor in combination with other treatments effective against certain neoplastic conditions, in particular, anti-cancer treatment with a MEK inhibitor or a b-RAF inhibitor, or a KRAS inhibitor.

### BACKGROUND

Cancer cell quiescence, effectively a cell in a state of sleep, has been recognized recently as a major mechanism of the resistance of cancer cells to treatments and for providing a pathway for disease recurrence. Quiescence, alternatively called cellular dormancy, is due to arrest of a cell in the G₀ phase of the cell cycle. Typically, a cell enters a cell cycle from gap phase 1 (G₁). After a synthesis phase (S) and a short pre-mitotic interval (G₂), the cell divides by mitosis (M) followed by a return to G₁. Instead of G₁, however, a cell can enter cellular dormancy, also termed quiescence, designated as the G₀ phase. Cancer cells can enter this reversible, quiescent G₀ state from which they could resume cycling (Coller H A, Sang L, and Roberts J M (2006) A new description of cellular quiescence, PLoS Biology 4, e83).

A fraction of a population of cells naturally may be in a quiescent state at any given time and remain quiescent for an indeterminate period until receipt of a signal to enter the cell division cycle. The proportion of cancer cells in quiescent state within a cell population, for example within a tumor, may be increased by environmental factors, such as lack of nutrients, hypoxia, high concentration of reactive oxygen species, etc. Cells may also be induced into the quiescent state by the action of a drug substance, as in pharmacological quiescence.

The energy and nutrient requirements of a quiescent cell are reduced relative to a dividing cell. Most current cancer therapies target dividing cells, and therefore a cancer cell must be in the cell division cycle for such treatments to affect it. Accordingly, a quiescent cancer cell is resistant to treatments that affect one or more cellular proliferation processes by means of damaging exposed DNA, interfering with DNA replication or repair, interfering with mitosis, or other mechanisms.

Both anticancer therapeutics and radiation treatments produce adverse effects. Consequently, doses and duration of treatment are limited by toxicity and lower effective doses and/or shorter treatment durations are highly desirable. Upon reduction in doses or discontinuation of treatment, however, the surviving quiescent cancer cells can cause cancer recurrence upon re-entry to the cell cycle, the timing of which cannot be predicted. Further, metastatic cancer cells in the bloodstream may experience a period of quiescence while they adapt to their new microenvironment (Chaffer C L and Weinberg R A (2011). A perspective on cancer cell metastasis, Science 331, 1559-1564). Quiescent cancer cells degrade their polyribosomes, thus blocking translation and reducing total RNA and protein content. These shrunk cancer cells may be able to enter the bores of capillaries (approximately 8 μm diameter) whereas cycling cancer cells are usually much larger (20-30 μm).

Accordingly, the existence of a population of quiescent cancer cells within a neoplasm has been recognized as an obstacle to successful and durable treatment (Jackson R C (1989) The problem of the quiescent cancer cell, Advances in Enzyme Regulation 29, 27-46). Evidence for resistance of quiescent cancer cells derived from various cancer types and to various anti-cancer treatments has been reported. Yet, despite a growing appreciation of the importance of cancer cell quiescence, and research and development efforts, this issue remains unaddressed clinically in general and in particular as it relates to the survival mechanisms of cancer cells evolved or activated in response to therapeutic disruption of a signaling pathway (Recasens, A., and Munoz, L. (2019) Targeting Cancer Cell Dormancy, Trends in Pharmacological Sciences 40, 128-141; Zhang, J., Si, J., et al. (2019) Research progress on therapeutic targeting of quiescent cancer cells, Artificial Cells, Nanomedicine, and Biotechnology 47, 2810-2820).

DYRK1B/Mirk is a member of the Minibrain/DYRK family of kinases which mediates survival and differentiation in certain normal tissues. (Kentrup H, Becker W, Heukelbach J, Wilmes A, Schurmann A, Huppertz C, Kainulainen H, and Joost H G (1996) Dyrk, a dual specificity protein kinase with unique structural features whose activity is dependent on tyrosine residues between subdomains VII and VIII, Journal of Biological Chemistry 271, 3488-3495; Becker W, Weber Y, Wetzel K, Eirmbter K, Tejedor F J, and Joost H G (1998) Sequence characteristics, subcellular localization, and substrate specificity of DYRK-related kinases, a novel family of dual specificity protein kinases, Journal of Biological Chemistry 273, 25893-25902). DYRK1B is expressed at detectable levels in skeletal muscle cells and testes. Knockout of DYRK1B caused no evident abnormal phenotype in mice even in developing muscle, suggesting that DYRK1B is not an essential gene for normal development. Supporting this interpretation, normal fibroblasts exhibited no alteration in survival after 20-fold depletion of DYRK1B kinase levels. Thus, DYRK1B does not appear to be an essential gene for survival of normal cells yet there is evidence that it is upregulated in certain malignant cancer cells in which DYRK1B is believed to mediate survival by retaining cancer cells in quiescent state. These unusual characteristics suggest that DYRK1B may be an attractive target for therapeutic intervention and in particular for anti-cancer therapy directly against quiescent cancer cells.

The KRAS (Kirsten Rat Sarcoma) protein is one of three human RAS GTPases and acts as a master switch of the MAPK pathway: KRAS→RAF→MEK→ERK. KRAS, unlike HRAS and NRAS, has preferential specificity for RAF over another RAS downstream effector protein, PI3K (Stalnecker, C. A., and Der, C. J. (2020) RAS, wanted dead or alive: Advances in targeting RAS mutant cancers, Science Signaling 13, eaay6013). KRAS is a proto-oncogene and its mutations are the most common mutations in pancreatic cancer (found in over 90% of cases), colon (over 40%), and non-small cell lung cancer (NSCLC) with adenocarcinoma histology (over 30%).

KRAS mutations result in the activation of the RAS-RAF-MEK-ERK or MAPK pathway. The MAPK pathway is regulated by RAS (KRAS, HRAS, NRAS) binding to B-RAF or RAF1, which are mitogen-activated protein kinases (MAP3Ks). MAP3Ks phosphorylate and activate MAP-ERK kinases 1 and 2 (MEK1/2), both of which phosphorylate and activate extracellular signal-regulated kinases (ERK1 and 2). The importance of the RAS-RAF-MEK-ERK pathway is demonstrated by the essential functions that ERK1/2 substrates regulate, including proliferation, survival, translation, lipid metabolism, transcription, and protein acetylation.

Anticancer agents that target RAS-RAF-MEK-ERK pathways include MEK inhibitors, b-Raf inhibitors, and most recently, inhibitors of both wild-type and mutant KRAS, among others. Multiple clinical trials have been conducted investigating the effects on cancers with different mutation profiles of single inhibitors targeting MEK, b-Raf, KRAS and certain KRAS mutants, or combinations of inhibitors targeting proteins within the same pathway or by inhibiting downstream proteins in the MEK-ERK and the PI3K-AKT-mTOR pathways, in combinations with an EGFR TKI therapy, etc. (Kim, Y. H. (2016) Dual inhibition of B-RAF and MEK in B-RAF-mutated metastatic non-small cell lung cancer, Journal of Thoracic Disease 8, 2369-2371; Lin, X., Liao, J., et al. (2020). Concurrent inhibition of ErbB family and MEK/ERK kinases to suppress non-small cell lung cancer proliferation, American Journal of Translational Research 12, 847-856; Sato, H., Yamamoto, H., et al. (2018) Combined inhibition of MEK and PI3K pathways overcomes acquired resistance to EGFR-TKIs in non-small cell lung cancer, Cancer Science 109, 3183-3196; Shrestha, N., Nimick, M., et al. (2019) Mechanisms of suppression of cell growth by dual inhibition of ALK and MEK in ALK-positive non-small cell lung cancer, Scientific Reports 9, 18842; Sunaga, N., Miura, Y., et al. (2019) Dual inhibition of MEK and p38 impairs tumor growth in KRAS-mutated non-small cell lung cancer, Oncology Letters, 17, 3569-3575). The effectiveness of a combination of therapeutic agents against cancers with different mutation profiles is, at present, impossible to predict (Broutin, S., Stewart, A., et al. (2016) Insights into significance of combined inhibition of MEK and m-TOR signalling output in KRAS mutant non-small-cell lung cancer, British Journal of Cancer 115, 549-552). Consequently, in addition to efficacy, the lack of generality of single agent or combination treatments across at least some of the mutational profiles is a major problem, given the multitude of mutational profiles and tumor heterogeneity observed in the clinic.

Therefore, there remains a need of improving therapeutic interventions treated with inhibitors of the MAPK pathway (also known as RAS-RAF-MEK-ERK pathway), particularly, aggressive, metastatic, and resistant cancers.

### SUMMARY OF THE INVENTION

The present invention provides compositions and methods for the treatment of neoplasms by the treatment with the combination of the therapeutic agents targeting the MAPK pathway and DYRK1 inhibitors.

Generally, the invention features a method of treating a neoplasm comprising: administering to a subject in need thereof a therapeutically effective amount of at least two or more agents comprising: (a) a first agent being a DYRK1 inhibitor; and (b) the second agent which is an inhibitor of MAPK pathway, such as a MEK inhibitor, a B-RAF inhibitor including a V600E B-RAF mutant, or a pan-KRAS inhibitor, a KRAS-mutant inhibitor, or other inhibitors of MAPK pathway, including but not limited to inhibitors of the KRAS mutants on codons 12, 13, and 61, such as G12C, G12D, G12S, G12V, G12A, G13D, and Q61H, wherein two or more agents can be administered sequentially or concomitantly.

In some embodiments, the neoplasm is a cancer or a population of cancer cells in vitro or in vivo. In some embodiments, the subject (e.g., human or a mammal subject) receiving the treatment is diagnosed with cancer (e.g., metastatic or pre-metastatic) with deregulated MAPK pathway and/or with a mutated KRAS. In some embodiments, the subject has been previously treated with a first-line therapy against cancers with deregulated MAPK pathways or against KRAS-mutated cancer. In some embodiments, the subject is treated, or has been treated, with two or more of MAPK pathway inhibitors sequentially or concomitantly.

In some embodiments, the combined treatment may result in improved outcomes, such as increased survival, reduction of severity, delay or elimination of recurrence, elimination of resistance, increase in treatment durability, or reduced side effects of the primary treatments (i.e., a MEK, B-RAF or KRAS inhibitor). In some embodiments, the second agent is administered at a lower dose for a shorter duration when administered as part of the combination as compared to a treatment with the agent alone. For example, in some embodiments, the EC₅₀ value of the MAPK pathway inhibitor is at least 20%, 30%, 40%, 50%, 60%, 70, 75%, 80%, 85%, 90%, 95% or lower in the combination treatment with a DYRK1 inhibitor when compared to the same treatment with a MAPK pathway inhibitor as a single agent, as determined, for example, in cell-based assays. In some embodiments, the combination treatment increases fraction of apoptotic cells in a treated population as compared to either agent alone, by at least 2-fold as determined, for example, by fraction of sub-G₀ phase cells in a FACS assay.

In one embodiment, the therapeutic agent effective against cancer cells is a DYRK1 inhibitor. In some embodiments, the DYRK1 inhibitor is a compound that inhibits activity of a DYRK1 kinase, either DYRK1A and/or DYRK1B (in vitro or in vivo), for example, with an IC₅₀ of 100 nM or lower in biochemical assays. In some embodiments, the DYRK1 inhibitor reduces the fraction of quiescent cancer cells (in vitro or in vivo) that would otherwise be found in the absence of such inhibitor, for example, by at least 20%, 30%, 40%, 50%, 60%, 70, 75%, 80%, 85%, 90%, 95% or higher. In some embodiments, the DYRK1 inhibitor inhibits both DYRK1A and DYRK1B. In some embodiments, the DYRK1 inhibitor is selective for DYRK1A and/or DYRK1B.

In one embodiment, the DYRK1 inhibitor is a compound of formula I (U.S. Pat. No. 9,446,044):

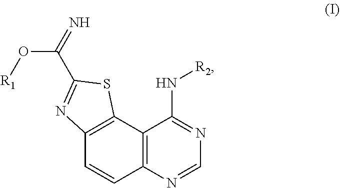

or a pharmaceutically acceptable salt or solvate thereof, wherein,
R₁ is a substituted or unsubstituted C₁₋₈ alkyl, a substituted or unsubstituted phenyl, or a substituted or unsubstituted benzyl;
R₂ is phenyl, optionally substituted with up to four groups independently selected from halo, CN, NO₂, NHC(O)C₁₋₄ alkyl, C₁₋₄ alkyl, OH, OC₁₋₄ alkyl, wherein two adjacent groups and their intervening carbon atoms may form a 5- to 6-membered ring containing one or more heteroatoms selected from N, O, or S.

In one embodiment, the compound of Formulas I-1 to I-7 is selected from:

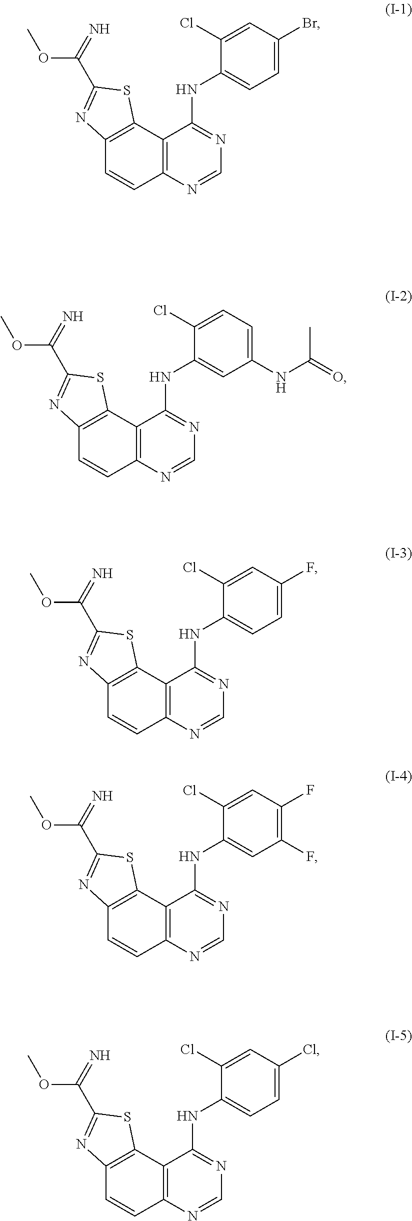 [SMILES: COC(=N)c1nc2ccc3ncnc(Nc4ccc(Br)cc4Cl)c3c2s1 ; COC(=N)c1nc2ccc3ncnc(Nc4cc(NC(C)=O)ccc4Cl)c3c2s1 ; COC(=N)c1nc2ccc3ncnc(Nc4ccc(F)cc4Cl)c3c2s1 ; COC(=N)c1nc2ccc3ncnc(Nc4cc(F)c(F)cc4Cl)c3c2s1 ; COC(=N)c1nc2ccc3ncnc(Nc4ccc(Cl)cc4Cl)c3c2s1]

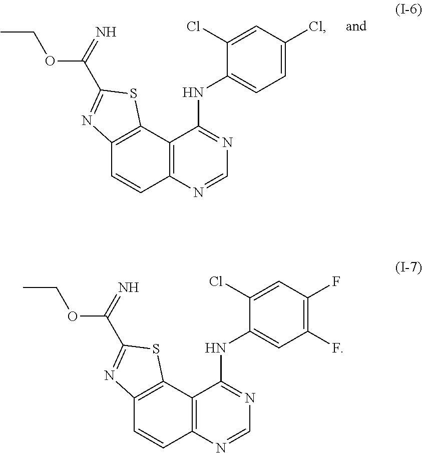 [SMILES: CCOC(=N)c1nc2ccc3ncnc(Nc4ccc(Cl)cc4Cl)c3c2s1 ; CCOC(=N)c1nc2ccc3ncnc(Nc4cc(F)c(F)cc4Cl)c3c2s1]

In one embodiment, the DYRK1 inhibitor is a compound of Formula II (U.S. Pat. No. 10,577,365):

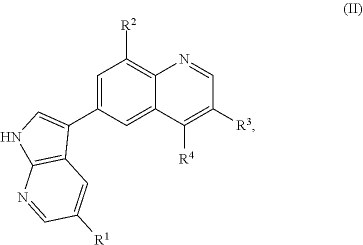

or a salt, stereoisomer, tautomer or N-oxide thereof,
wherein
R¹, R³, R⁴ are independently selected from the group consisting of
(i) H, halogen, CN, NO₂, C₁-C₆-alkyl, C₂-C₆-alkenyl, C₂-C₆-alkynyl; wherein each substitutable carbon atom in the aforementioned moieties is independently unsubstituted or substituted with one or more, same or different substituents R⁷;
(ii) C(═O)R⁵, C(═O)OR⁶, C(═O)SR⁶, C(═O)N(R^{6a})(R^{6b}), OR⁶, S(═O)ₙR⁶, S(═O)ₙN(R^{6a})(R^{6b}), S(═O)ₙOR⁶, N(R^{6a})(R^{6b}), N(R⁶)C(═O)R⁵, N(R⁶)C(═O)OR⁶, N(R⁶)C(═O)N(R^{6a})(R^{6b}), N(R⁶)S(═O)ₙR⁶, N(R⁶)S(═O)ₙN(R^{6a})(R^{6b}), N(R⁶)S(═O)ₙOR⁶;
(iii) a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R⁸;
   R² is selected from the group consisting of H, halogen, CN, NO₂, C₁-C₆-alkyl, C₁-C₆-haloalkyl, C₂-C₆-alkenyl, C₂-C₆-alkynyl, C₁-C₆-alkoxy and C₁-C₆-haloalkoxy;
   R⁵, R⁶, R^{6a}, R^{6b} are independently selected from the group consisting of H, C₁-C₆-alkyl, C₁-C₆-haloalkyl, C₂-C₆-alkenyl, C₂-C₆-alkynyl, C₁-C₆-alkylcarbonyl, wherein each substitutable carbon atom in the aforementioned moieties is independently unsubstituted or substituted with one or more, same or different substituents R⁹; and a 3- to 9-membered saturated, partially unsaturated or fully unsaturated carbocyclic or heterocyclic ring, wherein said heterocyclic ring comprises one or more, same or different heteroatoms selected from O, N or S, wherein said N and/or S-atoms are independently oxidized or non-oxidized, and wherein each substitutable carbon or heteroatom in the aforementioned cyclic moieties is independently unsubstituted or substituted with one or more, same or different substituents R¹⁰;
   R⁷ is selected from the group consisting of halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(═O)R⁵, C(═O)OR⁶, C(═O)SR⁶, C(═O)N(R^{6a})(R^{6b}), OR⁶, S(═O)ₙR⁶, S(═O)ₙN(R^{6a})(R^{6b}), S(═O)ₙOR⁶, N(R^{6a})(R^{6b}), N(R⁶)C(═O)R⁵, N(R⁶)C(═O)OR⁶, N(R⁶)C(═O)N(R^{6a})(R^{6b}), N(R⁶)S(═O)ₙ(R⁶), N(R⁶)S(═O)ₙN(R^{6a})(R^{6b}), N(R⁶)S(═O)ₙOR⁶; and a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned cyclic or bicyclic moieties is unsubstituted or substituted with one or more, same or different substituents R⁸;
   R⁸ is selected from the group consisting of halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkylcarbonyl, N(R^{6a})(R^{6b}), OR⁶ and S(═O)ₙR⁶;
   R⁹ is selected from the group consisting of halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkylcarbonyl, N(R^{11a})(R^{11b}), OR¹¹ and S(═O)ₙR¹¹; and a 3- to 9-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned cyclic moieties is unsubstituted or substituted with one or more, same or different substituents R¹⁰;
   R¹⁰ is selected from halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkylcarbonyl, N(R^{11a})(R^{11b}), OR¹¹ and S(═O)ₙR¹¹;
   R¹¹, R^{11a}, R^{11b} are independently selected from the group consisting of H, C₁-C₆-alkyl, C₂-C₆-alkenyl and C₂-C₆-alkynyl;
   and wherein
   n is 0, 1 or 2.

In one embodiment, R¹ is selected from the group consisting of
(i) H, halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl;
   wherein each substitutable carbon atom in the aforementioned moieties is independently unsubstituted or substituted with one or more, same or different substituents R⁷;
(ii) C(═O)R⁵, C(═O)OR⁶, C(═O)SR⁶, C(═O)N(R^{6a})(R^{6b}), OR⁶, S(═O)ₙR⁶, S(═O)ₙN(R^{6a})(R^{6b}), S(═O)ₙOR⁶, N(R^{6a})(R^{6b}), N(R⁶)C(═O)R⁵, N(R⁶)C(═O)OR⁶, N(R⁶)C(═O)N(R^{6a})(R^{6b}), N(R⁶)S(═O)ₙR⁶, N(R⁶)S(═O)ₙN(R^{6a})(R^{6b}), N(R⁶)S(═O)ₙOR⁶;
   preferably R¹ is selected from the group consisting of H, halogen, CN, NO₂, C₁-C₃-alkyl, C₂-C₃-alkenyl, C₂-C₃-alkynyl and C(═O)N(R^{6a})(R^{6b});
   wherein each substitutable carbon atom in the aforementioned moieties is independently unsubstituted or substituted with one or more, same or different substituents R⁷;
   more preferably R¹ is selected from the group consisting of H, halogen, CN, NO₂, C₁-C₃-alkyl, C₂-C₃-alkenyl and C₂-C₃-alkynyl;
   wherein each substitutable carbon atom in the aforementioned moieties is independently unsubstituted or substituted with one or more, same or different substituents R⁷;
   and wherein all other substituents have the meaning as defined above.

In another embodiment, R² is selected from the group consisting of H, halogen, CN, NO₂, C₁-C₂-alkyl, vinyl, C₁-C₂-alkoxy and C₁-C₂-haloalkoxy; and wherein all other substituents have the meaning as defined above.

In another embodiment R³ is selected from the group consisting of:
(i) H, halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl;
   wherein each substitutable carbon atom in the aforementioned moieties is independently unsubstituted or substituted with one or more, same or different substituents R⁷;
(ii) C(═O)R⁵, C(═O)OR⁶, C(═O)SR⁶, C(═O)N(R^{6a})(R^{6b}), OR⁶, S(═O)ₙR⁶, S(═O)ₙN(R^{6a})(R^{6b}), S(═O)ₙOR⁶, N(R^{6a})(R^{6b}), N(R⁶)C(═O)R⁵, N(R⁶)C(═O)OR⁶, N(R⁶)C(═O)N(R^{6a})(R^{6b}), N(R⁶)S(═O)ₙR⁶, N(R⁶)S(═O)ₙN(R^{6a})(R^{6b}), N(R⁶)S(═O)ₙOR⁶;
   preferably R³ is selected from the group consisting of H, halogen, CN, NO₂, N(R^{6a})(R^{6b}), N(R⁶)C(═O)R⁵; and wherein all other substituents have the meaning as defined above.

In another embodiment, R⁴ is selected from the group consisting of H, halogen, N(R^{6a})(R^{6b}), C₁-C₆-alkyl, C₂-C₆-alkenyl, C₂-C₆-alkynyl, wherein each substitutable carbon atom in the aforementioned moieties is independently unsubstituted or substituted with one or more, same or different R⁷; and a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R⁸; and wherein all other substituents have the meaning as defined above.

In another embodiment, R⁵, R⁶, R^{6a} and R^{6b} are independently from each other selected from the group consisting of H, C₁-C₅-alkyl, C₂-C₅-alkenyl, C₂-C₅-alkynyl, wherein each substitutable carbon atom in the aforementioned moieties is independently unsubstituted or substituted with one or more, same or different substituents R⁹; and a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned cyclic moieties is independently unsubstituted or substituted with one or more, same or different substituents R¹⁰.

In another embodiment, R⁷ is selected from the group consisting of halogen, CN, NO₂, C₁-C₅-alkyl, C₁-C₅-haloalkyl, C₂-C₅-alkenyl, C₂-C₅-haloalkenyl, C₂-C₅-alkynyl, C₂-C₅-haloalkynyl, OR⁶, N(R^{6a})(R^{6b}); and a 5- to 6-membered saturated, partially unsaturated or fully unsaturated carbocyclic or heterocyclic ring and a 8- to 9-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R⁸.

In another embodiment, R⁸ is selected from the group consisting of C₁-C₃-alkyl, C₂-C₃-alkenyl, C₁-C₃-alkylcarbonyl, C₂-C₃-alkynyl and N(R^{6a})(R^{6b}).

In another embodiment, R⁹ is selected from the group consisting of halogen, C₁-C₄-alkyl, C₂-C₄-alkenyl, C₂-C₄-alkynyl, N(R^{11a})(R^{11b}) and a 5- to 6-membered saturated, partially unsaturated or fully unsaturated carbocyclic or heterocyclic ring, wherein said heterocyclic ring comprises one or more, same or different heteroatoms selected from O, N or S, wherein said N and/or S-atoms are independently oxidized or non-oxidized, and wherein each substitutable carbon or heteroatom in the aforementioned cyclic moiety is independently unsubstituted or substituted with one or more, same or different substituents R¹⁰.

In another embodiment, R¹⁰ is selected from the group consisting of halogen, C₁-C₃-alkyl, C₂-C₃-alkenyl, C₁-C₃-alkylcarbonyl, C₂-C₃-alkynyl and N(R^{11a})(R^{11b}).

In yet another embodiment, R¹¹, R^{11a} and R^{11b} are independently selected from the group consisting of H, C₁-C₃-alkyl, C₂-C₃-alkenyl and C₂-C₃-alkynyl.

In another embodiment, the DYRK1 inhibitor is a compound of formula II, or a salt, stereoisomer, tautomer or N-oxide thereof, wherein

R² is selected from the group consisting of H, F, or Cl;

### R³ is H.

In one embodiment, the compound of formula II is:

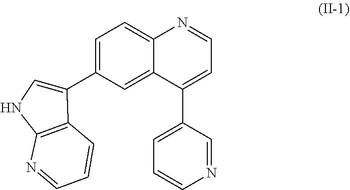 [SMILES: c1cncc(-c2ccnc3ccc(-c4c[nH]c5ncccc45)cc23)c1]

In another embodiment, the methods of the invention further provide (c) administering to the subject another cancer therapy, for example, radiation therapy or other cancer treatment. For example, the methods of the invention comprise: administering to a subject in need thereof a therapeutically effective amount of (a) DYRK1 inhibitor, such as of Formulas I-1 to I-7, of Formula II or another DYRK1-selective inhibitor; (b) a MAPK pathway inhibitor; and (c) radiation therapy; each therapy being administered sequentially or concomitantly. For example, in some embodiments, the subject is first treated with radiation therapy, whereupon the subject is administered a DYRK1 inhibitor, alone or in combination with a MEK, a b-Raf, and/or a KRAS inhibitor. In some embodiments, the subject is co-administered (a) a DYRK1 inhibitor, (b) a MAPK pathway inhibitor and, optionally (c) radiation therapy. In some embodiments, a −MAPK pathway inhibitor is a compound that inhibits activity of a wild-type or a mutant of a truncated MEK kinase (in vitro or in vivo), for example, with the IC₅₀ of 100 nM or lower in biochemical assays. In some embodiments, a MAPK pathway inhibitor is a compound that inhibits activity of wild-type or a mutant of a KRAS kinase (in vitro or in vivo). All such compounds that have been or will be approved for the treatment of cancer, or compounds that otherwise demonstrate safety and efficacy in treating cancer in mammalian subject (e.g., mice, rats, dogs, monkeys, humans), or compounds that demonstrate efficacy against neoplastic cells in vitro. Many such compounds are known.

In one embodiment, the MAPK pathway inhibitor is a MEK inhibitor, either of MEK1 or of MEK2. In another embodiment, the MEK-RAS-MAPK pathway inhibitor is an inhibitor of b-Raf and an inhibitor of b-Raf with a V600E mutation, such as, for example, vemurafenib, dabrafenib, encorafenib, or sorafenib. In a further embodiment, MEK-RAS-MAPK pathway inhibitor is an inhibitor of wild type or mutated (mutant) KRAS, including but not limited to KRAS mutants on codons 12, 13, and 61, such as G12C, G12D, G12V, G13D, and Q61H. In a further embodiment, a MEK inhibitor is, for example, trametinib, cobimetinib, binimetinib, selumetinib, PD-325901, CH5126766 (RO5126766), or CI-1040. In yet another embodiment, a KRAS-mutant inhibitor is a pan-RAS inhibitor such as BI 1701963 or BBP-454, affecting this inhibition by means of inhibiting the formation or function of a KRAS-SOS1 complex or by any other means, or an inhibitor of certain mutant KRAS proteins, such as sotorasib (AMG 510), MRTX849, MRTX1257, ARS-853, ARS-3248 (JNJ 74699157). In another embodiment, the MEK-RAS-MAPK pathway inhibitor is a combination of two or more inhibitors of MEK, b-Raf, and KRAS.

In another embodiment, the neoplasm being treated is a cancer, for example, colon, colorectal, breast, brain, prostate, pancreatic or ovarian cancers, acute lymphocytic leukemia (ALL), acute myelogenous leukemia (AML), juvenile myelomonocytic leukemia (JMML), non-small cell lung cancer (NSCLC), small cell lung cancer (SCLC), lymphoma, melanoma, or myeloproliferative syndrome (MPS), osteosarcoma, neuroblastoma or glioblastoma. In further embodiments, the cancer is primary or metastatic. In yet further embodiments, the cancer is of the type represented by the cell line types shown in the Examples. In some embodiments, the subject having cancer possesses a mutation in the KRAS gene having an increased risk of cancer and/or resistance to certain MEK and/or KRAS mutant inhibitors.

The embodiments described here are illustrative and are not meant to be limiting with regard to additional combination components, routes and order of administration, patient type (previously untreated or previously treated, absence or presence of co-morbid conditions, age, etc.), or stage of patient's disease, type of MAPK pathway inhibitor, etc.

### BRIEF DESCRIPTION OF THE DRAWINGS

FIG. 1 shows a schematic diagram of a mitotic cycle of a eukaryotic cell.

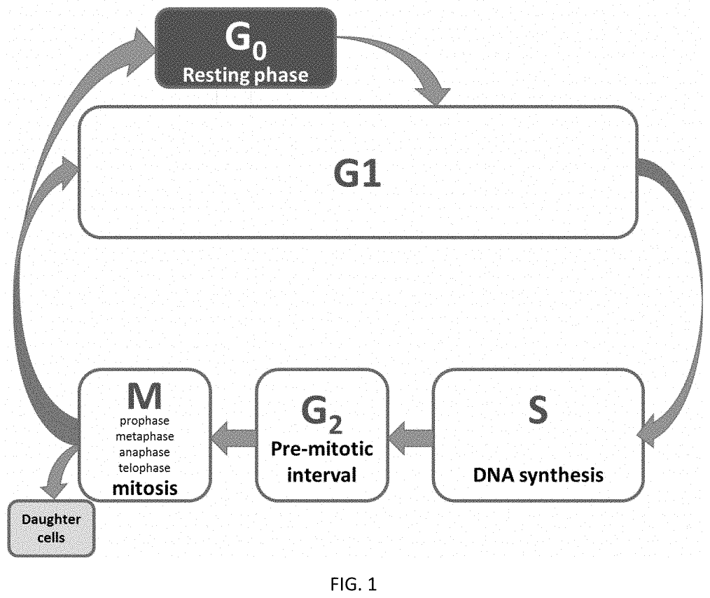

FIG. 2 shows a schematic diagram of a mitotic cycle of a eukaryotic cancer cell annotated to indicate the stages of the cell cycle upon which the available anti-cancer therapeutic agents are believed act.

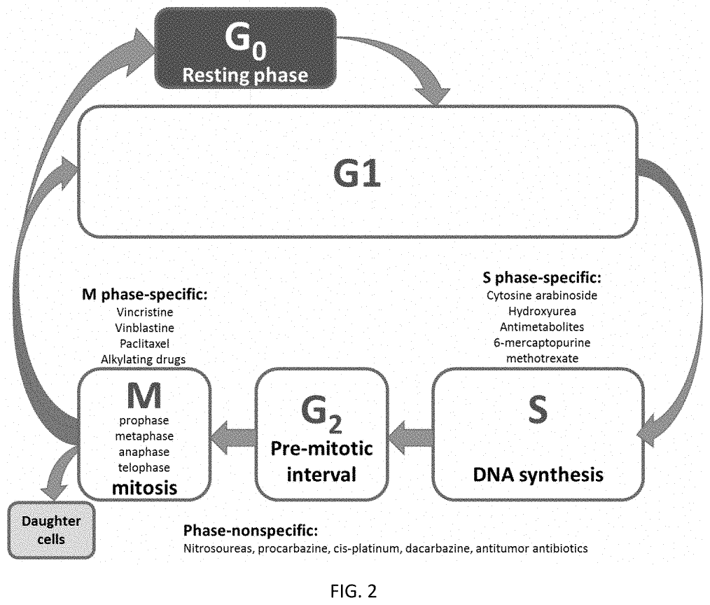

FIG. 3 shows effect of combination of selumetinib and Compound I-7 (0, 3, and 6 μM) on the growth of NSCLC A549 cells (KRAS-G12S).

FIG. 4 shows the effect of combination of selumetinib and Compound I-7 (0, 2.5, and 5 μM) on the growth of ovarian cancer OVCAR cells (wild KRAS).

FIG. 5 shows the effect of combination of trametinib and Compound I-7 (0, 2.2, and 6.7 μM) on the growth of NSCLC A549 cells (KRAS-G12S).

FIG. 6 shows the effect of combination of trametinib and Compound I-7 (0, 0.12, and 3.3 μM) on the growth of NSCLC H23 cells (KRAS-G12C).

FIG. 7 shows the effect of combination of trametinib and Compound I-7 (0, 5, and 10 μM) on the growth of 3D spheroids of NSCLC H23 cells (KRAS-G12C).

FIG. 8 shows the effect of combination of MRTX-849 and Compound I-7 (0, 2.5, and 5 μM) on the growth of NSCLC H2122 cells (KRAS-G12C).

FIG. 9 shows the effect of combination of AMG-510 and Compound I-7 (0, 2.5, and 5 μM) on the growth of NSCLC H2122 cells (KRAS-G12C).

FIG. 10 shows FACS analyses of cell cycle distribution of colon cancer SW620 cells (G12V) incubated for 24 hours in Panel A: FBS+ media; Panel B: FBS− media; Panel C: FBS+media with 5 μM Compound I-7; Panel D: FBS+ media with 20 nM trametinib; Panel D: FBS+media with 5 μM Compound I-7 and 20 nM trametinib.

FIG. 11 FACS analyses of cell cycle distribution of H2122 NSCLC cells (KRAS G12C) incubated for 48 hours in Panel A: FBS+ media; Panel B: FBS+ media with 5 μM Compound I-7; Panel C: FBS+ media with 100 nM MRTX 849; Panel D: FBS+ media with 5 μM Compound I-7 and 100 nM MRTX 849.

FIG. 12 FACS analyses of cell cycle distribution of H2122 NSCLC cells (KRAS G12C) incubated for 48 hours in Panel A: FBS+ media; Panel B: FBS+ media with 5 μM Compound I-7; Panel C: FBS+ media with 100 nM AMG 510; Panel D: FBS+ media with 5 μM Compound I-7 and 100 nM AMG-510.

FIG. 13 shows Western blot analysis showing expression levels of DYRK1B, phosphorylated T10202-MAPK, total MAPK, and β-actin in SW620 cells after 48-hour treatment with 20 nM of trametinib and 5 μM of Compound I-7 as indicated.

FIG. 14 shows the effect of combination of AMG-510 and Compound II-1 (0, 0.25 μM) on the growth of NSCLC H358 cells (KRAS-G12C).

FIG. 15 shows the effect of combination of MRTX-749 and Compound II-1 (0, 0.25 μM) on the growth of NSCLC H358 cells (KRAS-G12C).

FIG. 16 shows the effect of combination of MRTX-749 and Compound II-1 (0, 1 μM) on the growth of NSCLC H1975 cells (wild-type KRAS).

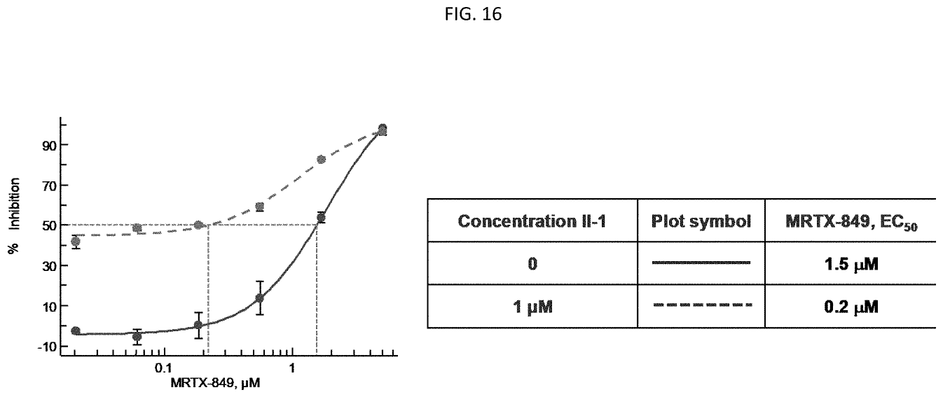

FIG. 17 shows the effect of combination of selumetinib and Compound II-1 (0, 1 μM) on the growth of NSCLC H1975 cells (wild-type KRAS).

### DETAILED DESCRIPTION OF THE INVENTION

### Definitions and Examples of their Use in the Methods of the Invention

In the present invention, an “alkyl” group is a saturated, straight or branched, hydrocarbon group, comprising from 1 to 8 carbon atoms (C₁₋₈ alkyl group), in particular from 1 to 6, or from 1 to 4 carbons atoms, unless otherwise indicated. Examples of alkyl groups having from 1 to 6 carbon atoms inclusive are methyl, ethyl, propyl (e.g., n-propyl, iso-propyl), butyl (e.g., tert-butyl, sec-butyl, n-butyl), pentyl (e.g., neo-pentyl), hexyl (e.g., n-hexyl), 2-methylbutyl, 2-methylpentyl and the other isomeric forms thereof. Alkyl groups may be unsubstituted or substituted by at least one group chosen from halogen atoms, cycloalkyl, heterocycloalkyl, aryl, heteroaryl, hydroxyl, alkoxyl, alkenyl, alkynyl, CN, nitro, and amino groups.

In the present invention, an “alkenyl” group is a straight or branched hydrocarbon group comprising at least one double carbon-carbon bond, comprising from 2 to 8 carbon atoms (unless otherwise indicated). Examples of alkenyl containing from 2 to 6 carbon atoms are vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl and the isomeric forms thereof.

Alkenyl groups may be unsubstituted, or substituted by at least one group chosen from halogen atoms, cycloalkyl, heterocycloalkyl, aryl, heteroaryl, hydroxyl, alkoxyl, alkenyl, alkynyl, CN, nitro, and amino groups.

In the present invention, an “alkynyl” group is a straight or branched hydrocarbon group comprising at least one triple carbon-carbon bond, comprising from 2 to 8 carbon atoms.

Alkynyl groups may be substituted by at least one group chosen from halogen atoms, cycloalkyl, heterocycloalkyl, aryl, heteroaryl, hydroxyl, alkoxyl, alkenyl, alkynyl, CN, nitro, and amino groups.

In the present invention, an “aryl” group is an aromatic hydrocarbon cycle, comprising from 5 to 14 carbon atoms. Most preferred aryl groups are mono- or bi-cyclic and comprises from 6 to 14 carbon atoms, such as phenyl, alpha-naphtyl, 3-naphtyl, antracenyl, preferably phenyl. “Aryl” groups also include bicycles or tricycles comprising an aryl cycle fused to at least another aryl, heteroaryl, cycloalkyl or heterocycloalkyl group, such as benzodioxolane, benzodioxane, dihydrobenzofurane or benzimidazole. Aryl groups may be unsubstituted, or substituted by at least one (e.g. 1, 2 or 3) group chosen from halogen atoms, cycloalkyl, heterocycloalkyl, aryl, heteroaryl, hydroxyl, alkoxyl, alkenyl, alkynyl, CN, nitro and amino groups. In addition, aryl groups may be substituted by adjacent substituents which can, taken together with the carbon atom to which they are attached, form a 5- to 6-membered ring which may contain one or more heteroatom(s) selected from N, O, and S.

In the present invention, a “halogen atom” or “halo” is a Cl, Br, F, or I atom.

In the present invention, an “alkoxyl” group is an alkyl group linked to the rest of the molecule through an oxygen atom, of the formula O-alkyl.

In the present invention, an “amino” group is a NH₂, NH-alkyl, or N(alkyl)₂ group.

In the present invention, a “heteroaryl” group is an aryl group whose cycle is interrupted by at least at least one heteroatom, for example a N, O, or S atom, such as thiophene or pyridine. Heteroaryl groups may be unsubstituted, or substituted by at least one (e.g. 1, 2 or 3) group chosen from halogen atoms, cycloalkyl, heterocycloalkyl, aryl, heteroaryl, hydroxyl, alkoxyl, alkenyl, alkynyl, CN, nitro, and amino groups. In addition, heteroaryl groups may be substituted by adjacent substituents which can, taken together with the carbon atom to which they are attached, form a 5- to 6-membered ring which may contain one or more heteroatom(s) selected from N, O, and S.

In the present invention, a “cycloalkyl” denotes a saturated alkyl group that forms one cycle having preferably from 3 to 14 carbon atoms, and more preferably 3 to 8 carbon atoms, such as cyclopropyl, cyclobutyl, cyclopentyl, cyclohexyl, cycloheptyl, and cyclooctyl. cycloalkyl groups may be unsubstituted, or substituted by at least one (e.g. 1, 2 or 3) group chosen from halogen atoms, cycloalkyl, heterocycloalkyl, aryl, heteroaryl, hydroxyl, alkoxyl, alkenyl, alkynyl, CN, nitro and amino groups. In addition, cycloalkyl groups may be substituted by adjacent substituents which can, taken together with the carbon atom to which they are attached, form a 5- to 6-membered ring which may contain one or more heteroatom(s) selected from N, O, and S.

In the present invention, a “heterocycloalkyl” group is a cycloalkyl group comprising at least one heteroatom, such as pyrrolidine, tetrahydrothiophene, tetrahydrofuran, piperidine, pyran, dioxin, morpholine or piperazine. A heterocycloalkyl group may in particular comprise from four to fourteen carbon atoms, such as morpholinyl, piperidinyl, pyrrolidinyl, tetrahydropyranyl, dithiolanyl. heterocycloalkyl groups may be unsubstituted or substituted by at least one group chosen from halogen atoms, cycloalkyl, heterocycloalkyl, aryl, heteroaryl, hydroxyl, alkoxyl, alkenyl, alkynyl, CN, nitro, and amino groups. In addition, heterocycloalkyl groups may be substituted by adjacent substituents which can, taken together with the carbon atom to which they are attached, form a 5- to 6-membered ring which may contain one or more heteroatom(s) selected from N, O, and S.

As used herein, a “neoplasm” means an abnormal mass of tissue that results from neoplasia. “Neoplasia” means a process of an abnormal proliferation of cells. In some embodiments of the invention, a neoplasm is a solid cancer, or alternately a hematopoietic cancer. The neoplasia may be benign, pre-malignant, or malignant. The term neoplasm encompasses mammalian cancers, in some embodiments, human cancers, and carcinomas, sarcomas, blastomas of any tissue (for example adenocarcinomas, squamous cell carcinomas, osteosarcomas, etc.), germ cell tumors, glial cell tumors, lymphomas, leukemias, including solid and lymphoid cancers, kidney, breast, lung, head and neck, bladder, colon, ovarian, prostate, rectal, pancreatic, stomach, brain, head and neck, skin, uterine, cervical, testicular, esophagus, thyroid, liver cancers, biliary cancer, and cancer of the bone and cartilaginous tissue, including non-Hodgkin's lymphomas (e.g., Burkitt's, Small Cell, and Large Cell lymphomas) and Hodgkin's lymphoma, leukemia, multiple myeloma, and myelodysplastic syndrome.

As used herein, the terms “treat,” “treating,” or “treatment,” mean to counteract a medical condition (e.g., cancer) to the extent that the medical condition is improved according to a clinically acceptable standard. Improvement in cancer can include: 1) reduced rate of tumor growth (tumor growth inhibition), 2) tumor shrinkage (regression), 3) decreased amount of the therapeutics required to achieve the tumor shrinkage, 4) remission, whether partial or total, 5) reduction in metastases, 6) prolonging progression free survival, and 7) delay or elimination of recurrence. In certain embodiments of the invention, treating includes achieving, partially or substantially, one or more of the following results: partially or totally reducing the cancer mass, or volume, or the malignant cell count; ameliorating or improving a clinical symptom or indicator associated with solid cancers or hematopoietic cancers; delaying, inhibiting, or preventing the progression of solid cancers or hematopoietic cancers; or partially or totally delaying, inhibiting or preventing the onset or development of solid cancers or hematopoietic cancers. “Treatment” also can mean prolonging progression free survival (PFS) or prolonging survival in general as compared to expected PFS or survival without treatment or compared to standard-of-care treatment.

Treating includes prophylactic or preventative treatment. “Prophylactic treatment” refers to treatment before appearance or re-appearance of clinical symptoms of a target disorder to prevent, inhibit, or reduce its occurrence, severity, or progression.

As used herein, an “effective amount” refers to an amount of a therapeutic agent or a combination of therapeutic agents that is therapeutically or prophylactically sufficient to effectuate the desired improvement in the targeted disorder. Examples of effective amounts typically range from about 0.0001 mg/kg of body weight to about 500 mg/kg of body weight per single administered dose, such doses being administered once or over a period of time. An exemplary range is from about 0.0001 mg/kg of body weight to about 5 mg/kg per dose. In other examples, the range can be from about 0.0001 mg/kg to about 5 mg/kg per single administered dose. In still other examples, effective amounts range from about 0.01 mg/kg of body weight to 50 mg/kg of body weight per single administered dose, or from 0.01 mg/kg of body weight to 0.1 mg/kg, 0.5 mg/kg, 1 mg/kg, 2 mg/kg, 3 mg/kg, 4 mg/kg, 5 mg/kg, 6 mg/kg, 10 mg/kg, 20 mg/kg, 25 mg/kg, 30 mg/kg, or 40 mg/kg of body weight per single administered dose. For agents of known clinical use, an example of an effective dose is that amount approved of by a regulatory agency for treatment of an indication.

As used herein, the term “subject” refers to a mammal, for example a human, but can also mean an animal in need of veterinary treatment, e.g., companion animals (e.g., dogs, cats, and the like), farm animals (e.g., cows, sheep, pigs, horses, and the like), and laboratory animals (e.g., rats, mice, guinea pigs, and the like).

As used herein, the term “therapeutic agent” means any chemical molecule used or contemplated for use or investigated for use in cancer treatment, including cytotoxic, cytostatic, or targeted agents, whether small molecules, or peptides, or antibodies, or oligonucleotides, irrespective of mechanism of action. As used herein, the terms “therapeutic” or “therapeutic agent” refer to either the active pharmaceutical ingredient (API) or its pharmaceutically acceptable salt or hydrate (solvate), or a drug product containing the therapeutic agent, however formulated, and whether API is amorphous or crystalline and of whatever polymorphic form. Formulation means a combination of an active pharmaceutical ingredient (API, drug substance) or ingredients (APIs) combined with excipients and/or delivery vehicle to make an administrable dosage form (drug product).

The therapeutic agents of the invention are generally administered with a pharmaceutically acceptable carrier, with respect to standard pharmaceutical practice (such as described in Remington: The Science and Practice of Pharmacy, 21^{st} Edition, Lippincott Williams & Wilkins). Accordingly, a further object of this invention relates to a pharmaceutical compositions defined herein and pharmaceutically acceptable carriers.

As used herein, the term “inhibitor” means any composition that reduces the activity of an enzyme. An example of an inhibitor is a chemical molecule. A measure of the potency of an inhibitor is its “50% inhibitory concentration” (IC₅₀). IC₅₀ concentration or IC₅₀ value is the concentration of an inhibitor at which 50% of the enzymatic activity is inhibited by the inhibitor. Methods for the determination of IC₅₀ values, for example, of kinase inhibitors are known to persons of ordinary skill in the art and include direct and indirect functional assays, such as the HotSpot™ kinase assay technology (Reaction Biology Corporation, Malvern, Pa., www.reactionbiology.com) or competition binding assays, such as KINOMEscan® (Eurofins DiscoverX Corporation, Freemont, Calif., www.discoverx.com). Thus, in certain embodiments, the IC₅₀ of a DYRK1 inhibitor or a MAPK pathway inhibitor is 100 mM or less, 75 mM or less, 50 mM or less, 25 mM or less, or 10 mM or less.

A measure of the potency of a therapeutic agent against a cell line is its “50% effective concentration” (EC₅₀). EC₅₀ value is the concentration of a drug that produces half-maximal response, such as, for example, 50% cell growth inhibition or 50% reduction in cell viability. Methods for the determination of EC₅₀ values, for example, of kinase inhibitors are known to persons of ordinary skill in the art. For example, in some embodiments, the combination of a DYRK1 and a MAPK pathway inhibitor, EC₅₀ value of the MAPK pathway inhibitor is reduced by at least 10%, 20%, 30%, 40%, 50%, 60%, 70%, 80%, 90% or more.

As used herein, the term “quiescence” or “quiescent state” refers to the G₀ state of the cell cycle, as understood by the practitioners of the art.

A “quiescent neoplastic cell,” alternately referred to as a “quiescent cancer cell” means a cancer cell that exists in the quiescent, or G₀, state of the cell cycle. A “fraction of quiescent neoplastic cells” or “fraction of quiescent cancer cells”, as used herein, means the portion of a cancer cell population that exists in the G₀ state of the cell cycle. Determining the fraction of quiescent neoplastic cells includes characterizing a cell population by distribution of its constituent cells within the stages of the cell cycle. The fraction of cells in the G₀ state (i.e., quiescent neoplastic cells) is quantified relative to the total cell population. The fraction may be expressed as a percentage of the total cell population (i.e. (number of quiescent cells divided by total cells in cell population) multiplied by 100). Characterization of the cell population by distribution of its constituent cells within the stages of the cell cycle may be achieved by techniques known to persons of ordinary skill in the art, and may include analysis by DNA and/or RNA content distribution within the cell cycle using flow cytometry methods, for example, fluorescence-activated cell sorting (FACS).

As used herein, the term DYRK1 inhibitor refers to the inhibitor of either DYRK1B or DYRK1A, or a dual inhibitor of DYRK1A and DYRK1B.

Examples of DYRK1 inhibitors, both DYRK1A and DYRK1B, include both reversible and irreversible small molecule inhibitors including inhibitors of Formulas I-1-I-7 and II-1.

Examples of the MAPK pathway inhibitors, including MEK inhibitors, b-RAF inhibitors, and mutant KRAS inhibitors and wild-type KRAS inhibitors, include both reversible and irreversible small molecule inhibitors specified throughout this specification.

### DETAILED DESCRIPTION

The present invention provides compositions and methods for the treatment of neoplasms, in particular, by targeting cancers cells with DYRK1 inhibitors in combination with MAPK pathway inhibitors, (e.g., a MEK inhibitor or a b-RAF inhibitor, or a KRAS inhibitor therapeutic agents) that is particularly advantageous for certain neoplastic conditions, specifically, as an anti-cancer treatment with.

Generally, the invention features a method of treating a neoplasm comprising: administering to a subject in need thereof a therapeutically effective amount of (a) a first agent being a DYRK1 inhibitor; and (b) second agent which is a MEK inhibitor or a b-RAF inhibitor, or a KRAS inhibitor, wherein the two agents can be administered sequentially or concomitantly. In some embodiments, the neoplasm is a cancer or a population of cancer cells in vitro or in vivo. In some embodiments, the subject receiving the treatment is diagnosed with cancer (e.g., metastatic or pre-metastatic). In some embodiments, the subject has been treated previously with a first-line therapy against cancer. In some embodiments, the subject has been treated previously with second-line and/or other therapies. In some embodiments, the subject is treated, or has been treated, with radiation therapy. In some embodiments, the subject was treated with surgery, for example, to resect or debulk a tumor. In other embodiments, the subject's neoplasm has recurred. In some embodiments, the subject is treated, or has been treated, with two or more of: a MEK inhibitor, a b-RAF inhibitor, and a KRAS inhibitors, sequentially or concomitantly.

In some embodiments, the combined treatment may result in improved outcomes, such as increased survival, reduction of severity, delay or elimination of recurrence, reduced required dose of the primary treatments, or reduced side effects of the primary treatments (i.e., a MEK inhibitor, a b-RAF inhibitor, or a KRAS inhibitor). In some embodiments, the second agent is administered at lower dose and/or for a shorter duration when administered as part of the combination as compared to a treatment with the agent alone. For example, in some embodiments, the EC₅₀ value of a MEK inhibitor or a b-RAF inhibitor, or a KRAS inhibitor is at least 20%, 25%, 30%, 40%, 50%, 60%, 70%, 80% or 90% lower in the combination treatment with DYRK1 inhibitor when compared to the same treatment with a MEK inhibitor or a b-RAF inhibitor, or a KRAS inhibitor as a single agent, as determined, for example, in cell-based assays. In some embodiments, the combination treatment increases fraction of apoptotic cells in a treated cancer cell population as compared to either agent alone, by at least by 2-fold, 3-fold, 4-fold, 5-fold, or 10-fold as determined, for example, by fraction of sub-G₀ phase cells as determined by a FACS assay. In some embodiments, the fraction of quiescent cancer G₀ cells is decreased by at least 20%, 25%, 30%, 40%, 50% or more in the combination treatment with a DYRK1 inhibitor when compared to the same treatment with a MEK inhibitor or a b-RAF inhibitor, or a KRAS inhibitor as a single agent, as determined, for example, in cell-based assays. In some embodiments, the combination of a DYRK1 inhibitor and a second agents are additive. In some embodiments, the combination of a DYRK1 inhibitor and a second agents are synergetic, as determined, for example, in cell-based assays via Chou-Talalay method or other methods known those skilled in the art.

In one embodiment, the therapeutic agent is a DYRK1 inhibitor. In some embodiments, the DYRK1 inhibitor is a compound that inhibits activity of a DYRK1 kinase, either DYRK1A or DYRK1B (in vitro or in vivo), for example, with an IC₅₀ value of <100 nM, <90 nM, <80 nM, <70 nM, <60 nM, <50 nM, <40 nM, <30 nM, <20 nM, <10 nM, <5 nM or lower in biochemical assays. In some embodiments, the DYRK1 inhibitor reduces the fraction of quiescent cancer cells (in vitro or in vivo) in a population or a tumor that would otherwise be found in the absence of such inhibitor, for example, by at least 5%, 10%, 15%, 20%, 25%, 30%, 40%, 50%, or more. In some embodiments, the DYRK1 inhibitor inhibits both DYRK1A and DYRK1B. In some embodiments, the DYRK1 inhibitor is selective for DYRK1A, with ratio of DYRK1B IC₅₀ to DYRK1A IC₅₀ of 1000, 100, 50, 25, 10 to 1. In some embodiments, the DYRK1 inhibitor is selective for DYRK1B, with ratio of DYRK1A IC₅₀ to DYRK1B IC₅₀ of 1000, 100, 50, 25, 10 to 1. In some embodiments, the DYRK1 inhibitor is selective for DYRK1 by at least 4-fold, 5-fold, 10-fold, 20-fold, 50-fold, 100-fold as compared to DYRK2 and/or DYRK3 and/or DYRK4, as determined by ratios of IC₅₀ values. In some embodiments, the DYRK1 inhibitor is selective for DYRK1 by at least 4-fold, 5-fold, 10-fold, 20-fold, 50-fold, 100-fold, 500-fold, 1000-fold as compared to cyclin dependent kinases (CDKs) such as, for example, CDK2, CDK4, or CDK6, as determined by ratios of IC₅₀ values. In some embodiments, the DYRK1 inhibitor is selective for DYRK1 by at least 4-fold, 5-fold, 10-fold, 20-fold, 50-fold, 100-fold, 500-fold, 1000-fold as compared to GSK30, as determined by ratios of IC₅₀ values.

Examples of known DYRK1 inhibitors include: AZ191, DYRKi, harmine, ID-8, leucettine L41, NCGC00185981, INDY, ProINDY, TC-S 7004, and TG003. At least one known DYRK1 inhibitor, TC-S 7004, (US20120184562) is reported to be effective against both quiescent and proliferating cancer cells in vitro (Ewton D Z, Hu J, Vilenchik M, Deng X, Luk K C, Polonskaia A, Hoffman A F, Zipf K, Boylan J F, and Friedman E A. (2011) Inactivation of MIRK/DYRK1B kinase targets both quiescent and proliferating pancreatic cancer cells. Molecular Cancer Therapeutics 10: 2104-2114).

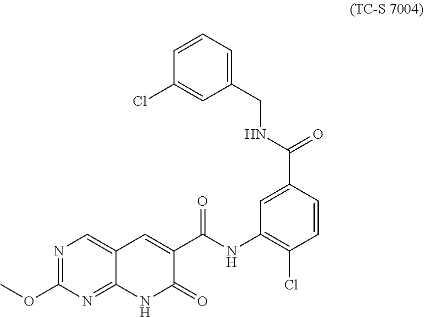 [SMILES: COc1ncc2cc(C(=O)Nc3cc(C(=O)NCc4cccc(Cl)c4)ccc3Cl)c(=O)[nH]c2n1]

In one embodiment, the DYRK1 inhibitor is a compound of formula I (U.S. Pat. No. 9,446,044):

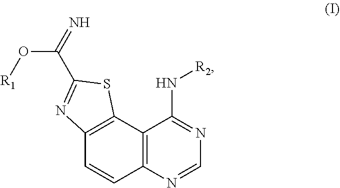

or a pharmaceutically acceptable salt or solvate thereof, wherein,
R₁ is a substituted or unsubstituted C₁₋₈ alkyl, a substituted or unsubstituted phenyl, or a substituted or unsubstituted benzyl;
R₂ is phenyl, optionally substituted with up to four groups independently selected from halo, CN, NO₂, NHC(O)C₁₋₄ alkyl, C₁₋₄ alkyl, OH, OC₁₋₄ alkyl, wherein two adjacent groups and their intervening carbon atoms may form a 5- to 6-membered ring containing one or more heteroatoms selected from N, O, or S.

In one embodiment, the compound of formula I is selected from:

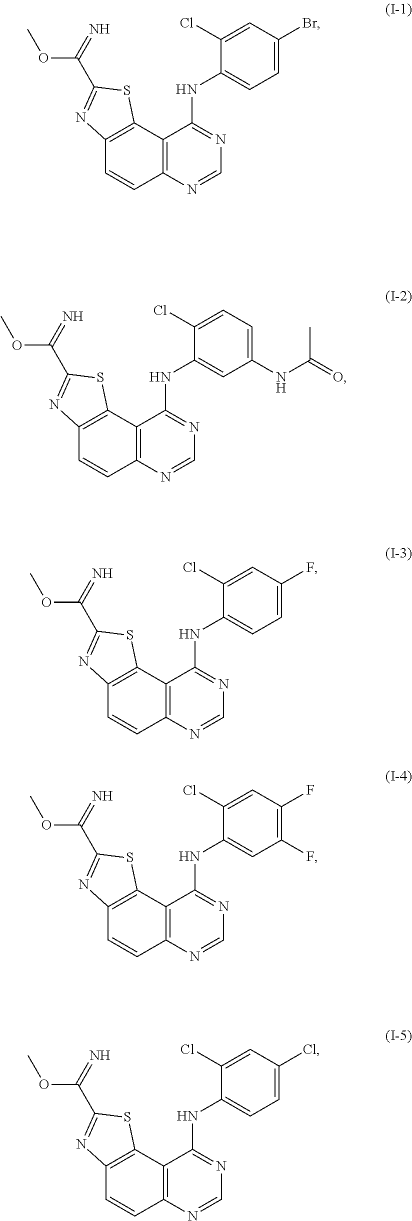 [SMILES: COC(=N)c1nc2ccc3ncnc(Nc4ccc(Br)cc4Cl)c3c2s1 ; COC(=N)c1nc2ccc3ncnc(Nc4cc(NC(C)=O)ccc4Cl)c3c2s1 ; COC(=N)c1nc2ccc3ncnc(Nc4ccc(F)cc4Cl)c3c2s1 ; COC(=N)c1nc2ccc3ncnc(Nc4cc(F)c(F)cc4Cl)c3c2s1 ; COC(=N)c1nc2ccc3ncnc(Nc4ccc(Cl)cc4Cl)c3c2s1]

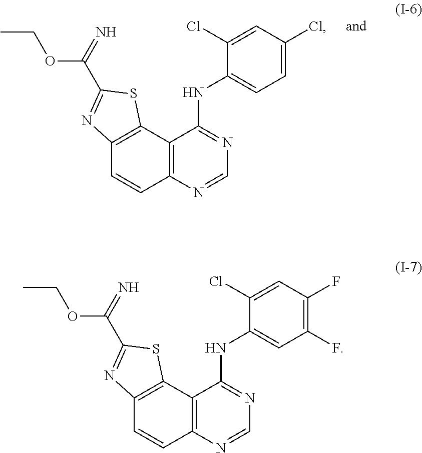 [SMILES: CCOC(=N)c1nc2ccc3ncnc(Nc4ccc(Cl)cc4Cl)c3c2s1 ; CCOC(=N)c1nc2ccc3ncnc(Nc4cc(F)c(F)cc4Cl)c3c2s1]

In one embodiment, the DYRK1 inhibitor is a compound of Formula II (U.S. Pat. No. 10,577,365):

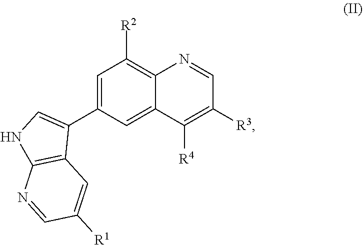

or a salt, stereoisomer, tautomer or N-oxide thereof,
wherein
R¹, R³, R⁴ are independently selected from the group consisting of
(iv) H, halogen, CN, NO₂, C₁-C₆-alkyl, C₂-C₆-alkenyl, C₂-C₆-alkynyl; wherein each substitutable carbon atom in the aforementioned moieties is independently unsubstituted or substituted with one or more, same or different substituents R⁷;
(v) C(═O)R⁵, C(═O)OR⁶, C(═O)SR⁶, C(═O)N(R^{6a})(R^{6b}), OR⁶, S(═O)ₙR⁶, S(═O)ₙN(R^{6a})(R^{6b}), S(═O)ₙOR⁶, N(R^{6a})(R^{6b}), N(R⁶)C(═O)R⁵, N(R⁶)C(═O)OR⁶, N(R⁶)C(═O)N(R^{6a})(R^{6b}), N(R⁶)S(═O)ₙR⁶, N(R⁶)S(═O)ₙN(R^{6a})(R^{6b}), N(R⁶)S(═O)ₙOR⁶;
(vi) a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R⁸;
   R² is selected from the group consisting of H, halogen, CN, NO₂, C₁-C₆-alkyl, C₁-C₆-haloalkyl, C₂-C₆-alkenyl, C₂-C₆-alkynyl, C₁-C₆-alkoxy and C₁-C₆-haloalkoxy;
   R⁵, R⁶, R^{6a}, R^{6b} are independently selected from the group consisting of H, C₁-C₆-alkyl, C₁-C₆-haloalkyl, C₂-C₆-alkenyl, C₂-C₆-alkynyl, C₁-C₆-alkylcarbonyl, wherein each substitutable carbon atom in the aforementioned moieties is independently unsubstituted or substituted with one or more, same or different substituents R⁹; and a 3- to 9-membered saturated, partially unsaturated or fully unsaturated carbocyclic or heterocyclic ring, wherein said heterocyclic ring comprises one or more, same or different heteroatoms selected from O, N or S, wherein said N and/or S-atoms are independently oxidized or non-oxidized, and wherein each substitutable carbon or heteroatom in the aforementioned cyclic moieties is independently unsubstituted or substituted with one or more, same or different substituents R¹⁰;
   R⁷ is selected from the group consisting of halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(═O)R⁵, C(═O)OR⁶, C(═O)SR⁶, C(═O)N(R^{6a})(R^{6b}), OR⁶, S(═O)ₙR⁶, S(═O)ₙN(R^{6a})(R^{6b}), S(═O)ₙOR⁶, N(R^{6a})(R^{6b}), N(R⁶)C(═O)R⁵, N(R⁶)C(═O)OR⁶, N(R⁶)C(═O)N(R^{6a})(R^{6b}), N(R⁶)S(═O)ₙ(R⁶), N(R⁶)S(═O)ₙN(R^{6a})(R^{6b}), N(R⁶)S(═O)ₙOR⁶; and a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned cyclic or bicyclic moieties is unsubstituted or substituted with one or more, same or different substituents R⁸;
   R⁸ is selected from the group consisting of halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkylcarbonyl, N(R^{6a})(R^{6b}), OR⁶ and S(═O)ₙR⁶;
   R⁹ is selected from the group consisting of halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkylcarbonyl, N(R^{11a})(R^{11b}), OR¹¹ and S(═O)ₙR¹¹; and a 3- to 9-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned cyclic moieties is unsubstituted or substituted with one or more, same or different substituents R¹⁰;
   R¹⁰ is selected from halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkylcarbonyl, N(R^{11a})(R^{11b}), OR¹¹ and S(═O)ₙR¹¹;
   R¹¹, R^{11a}, R^{11b} are independently selected from the group consisting of H, C₁-C₆-alkyl, C₂-C₆-alkenyl and C₂-C₆-alkynyl;
   and wherein
   n is 0, 1 or 2.

In one embodiment, R¹ is selected from the group consisting of
(iii) H, halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl;
   wherein each substitutable carbon atom in the aforementioned moieties is independently unsubstituted or substituted with one or more, same or different substituents R⁷;
(iv) C(═O)R⁵, C(═O)OR⁶, C(═O)SR⁶, C(═O)N(R^{6a})(R^{6b}), OR⁶, S(═O)ₙR⁶, S(═O)ₙN(R^{6a})(R^{6b}), S(═O)ₙOR⁶, N(R^{6a})(R^{6b}), N(R⁶)C(═O)R⁵, N(R⁶)C(═O)OR⁶, N(R⁶)C(═O)N(R^{6a})(R^{6b}), N(R⁶)S(═O)ₙR⁶, N(R⁶)S(═O)ₙN(R^{6a})(R^{6b}), N(R⁶)S(═O)ₙOR⁶;
   preferably R¹ is selected from the group consisting of H, halogen, CN, NO₂, C₁-C₃-alkyl, C₂-C₃-alkenyl, C₂-C₃-alkynyl and C(═O)N(R^{6a})(R^{6b});
   wherein each substitutable carbon atom in the aforementioned moieties is independently unsubstituted or substituted with one or more, same or different substituents R⁷;
   more preferably R¹ is selected from the group consisting of H, halogen, CN, NO₂, C₁-C₃-alkyl, C₂-C₃-alkenyl and C₂-C₃-alkynyl;
   wherein each substitutable carbon atom in the aforementioned moieties is independently unsubstituted or substituted with one or more, same or different substituents R⁷;
   and wherein all other substituents have the meaning as defined above.

In another embodiment, R² is selected from the group consisting of H, halogen, CN, NO₂, C₁-C₂-alkyl, vinyl, C₁-C₂-alkoxy and C₁-C₂-haloalkoxy; and wherein all other substituents have the meaning as defined above.

In another embodiment R³ is selected from the group consisting of:
(v) H, halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl;
   wherein each substitutable carbon atom in the aforementioned moieties is independently unsubstituted or substituted with one or more, same or different substituents R⁷;
(vi) C(═O)R⁵, C(═O)OR⁶, C(═O)SR⁶, C(═O)N(R^{6a})(R^{6b}), OR⁶, S(═O)ₙR⁶, S(═O)ₙN(R^{6a})(R^{6b}), S(═O)ₙOR⁶, N(R^{6a})(R^{6b}), N(R⁶)C(═O)R⁵, N(R⁶)C(═O)OR⁶, N(R⁶)C(═O)N(R^{6a})(R^{6b}), N(R⁶)S(═O)ₙR⁶, N(R⁶)S(═O)ₙN(R^{6a})(R^{6b}), N(R⁶)S(═O)ₙOR⁶;
   preferably R³ is selected from the group consisting of H, halogen, CN, NO₂, N(R^{6a})(R^{6b}), N(R⁶)C(═O)R⁵; and wherein all other substituents have the meaning as defined above.

In another embodiment, R⁴ is selected from the group consisting of H, halogen, N(R^{6a})(R^{6b}), C₁-C₆-alkyl, C₂-C₆-alkenyl, C₂-C₆-alkynyl, wherein each substitutable carbon atom in the aforementioned moieties is independently unsubstituted or substituted with one or more, same or different R⁷; and a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R⁸; and wherein all other substituents have the meaning as defined above.

In another embodiment, R⁵, R⁶, R^{6a} and R^{6b} are independently from each other selected from the group consisting of H, C₁-C₅-alkyl, C₂-C₅-alkenyl, C₂-C₅-alkynyl, wherein each substitutable carbon atom in the aforementioned moieties is independently unsubstituted or substituted with one or more, same or different substituents R⁹; and a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned cyclic moieties is independently unsubstituted or substituted with one or more, same or different substituents R¹⁰.

In another embodiment, R⁷ is selected from the group consisting of halogen, CN, NO₂, C₁-C₅-alkyl, C₁-C₅-haloalkyl, C₂-C₅-alkenyl, C₂-C₅-haloalkenyl, C₂-C₅-alkynyl, C₂-C₅-haloalkynyl, OR⁶, N(R^{6a})(R^{6b}); and a 5- to 6-membered saturated, partially unsaturated or fully unsaturated carbocyclic or heterocyclic ring and a 8- to 9-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R⁸.

In another embodiment, R⁸ is selected from the group consisting of C₁-C₃-alkyl, C₂-C₃-alkenyl, C₁-C₃-alkylcarbonyl, C₂-C₃-alkynyl and N(R^{6a})(R^{6b}).

In another embodiment, R⁹ is selected from the group consisting of halogen, C₁-C₄-alkyl, C₂-C₄-alkenyl, C₂-C₄-alkynyl, N(R^{11a})(R^{11b}) and a 5- to 6-membered saturated, partially unsaturated or fully unsaturated carbocyclic or heterocyclic ring, wherein said heterocyclic ring comprises one or more, same or different heteroatoms selected from O, N or S, wherein said N and/or S-atoms are independently oxidized or non-oxidized, and wherein each substitutable carbon or heteroatom in the aforementioned cyclic moiety is independently unsubstituted or substituted with one or more, same or different substituents R¹⁰.

In another embodiment, R¹⁰ is selected from the group consisting of halogen, C₁-C₃-alkyl, C₂-C₃-alkenyl, C₁-C₃-alkylcarbonyl, C₂-C₃-alkynyl and N(R^{11a})(R^{11b}).

In yet another embodiment, R¹¹, R^{11a} and R^{11b} are independently selected from the group consisting of H, C₁-C₃-alkyl, C₂-C₃-alkenyl and C₂-C₃-alkynyl.

In another embodiment, the DYRK1 inhibitor is a compound of formula II, or a salt, stereoisomer, tautomer or N-oxide thereof,

wherein
R² is selected from the group consisting of H, F, or Cl;

### R³ is H.

In one embodiment, the compound of formula II is:

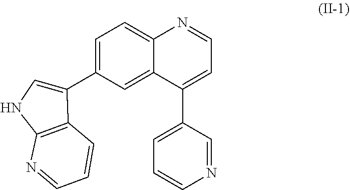 [SMILES: c1cncc(-c2ccnc3ccc(-c4c[nH]c5ncccc45)cc23)c1]

In another embodiment, the methods of the invention further provide: (c) administering to the subject another cancer therapy, for example, radiation therapy or other cancer treatment.

In one embodiment, the methods of the invention comprise: administering to a subject in need thereof a therapeutically effective amount of (a) a therapeutic agent of formula I; (b) a MEK inhibitor or a b-RAF inhibitor, or a KRAS inhibitor; and (c) radiation therapy; each therapy being administered sequentially or concomitantly. For example, in some embodiments, the subject is first treated with radiation therapy, whereupon the subject is administered a therapeutic agent of Formula I, alone or in combination with a MEK inhibitor or a b-RAF inhibitor, or a KRAS inhibitor. In some embodiments, the subject is co-administered (a) the therapeutic agent effective against quiescent cancer cells, (b) a MEK inhibitor or a b-RAF inhibitor, or a KRAS inhibitor and optionally (c) radiation therapy. In some embodiments, a MEK inhibitor or a b-RAF inhibitor, or a KRAS inhibitor is a compound that inhibits activity of wild type or one or more mutants in vitro or in vivo, for example, with the IC₅₀ of <100 nM, <90 nM, <80 nM, <70 nM, <60 nM, <50 nM, <40 nM, <30 nM, <20 nM, <10 nM, <5 nM, or lower in biochemical assays. In some embodiments, a MEK inhibitor or a b-RAF inhibitor, or a KRAS inhibitor is selective for the mutants over the wild type. In some embodiments, a MEK inhibitor or a b-RAF inhibitor, or a KRAS inhibitor is effective for the treatment of prevention of a neoplasm, including but not limited to, all such compounds approved for the treatment of cancer, compounds in clinical trials for the treatment of cancer, compounds that otherwise demonstrate efficacy in treating cancer in a mammalian subject (e.g., mouse, rats, dogs, monkeys, humans), and compounds that demonstrate efficacy against neoplastic cells in vitro. Many such compounds are known.

In one embodiment, the MAPK pathway inhibitor is a MEK inhibitor, either MEK1 or MEK2. In another embodiment, the MEK-RAS-MAPK pathway inhibitor is an inhibitor of b-Raf and an inhibitor of b-Raf with a V600E mutation, such as, for example, vemurafenib, dabrafenib, encorafenib, or sorafenib. In a further embodiment, MEK-RAS-MAPK pathway inhibitor is an inhibitor of wild type or mutated (mutant) KRAS, including but not limited to KRAS mutants on codons 12, 13, and 61, such as G12C, G12D, G12V, G13D, and Q61H. In a further embodiment, a MEK inhibitor is, for example, trametinib, cobimetinib, binimetinib, selumetinib, PD-325901, CH5126766 (RO5126766), or CI-1040. In yet another embodiment, a KRAS-mutant inhibitor is a pan-RAS inhibitor such as BI 1701963 or BBP-454, affecting this inhibition by means of inhibiting the formation or function of a KRAS-SOS1 complex or by any other means, or an inhibitor of certain mutant KRAS proteins, such as sotorasib (AMG 510), MRTX849, MRTX1257, ARS-853, ARS-3248 (JNJ 74699157). In another embodiment, the MEK-RAS-MAPK pathway inhibitor is a combination of two or more inhibitors of MEK, b-Raf, and KRAS.

In one embodiment, the neoplasm being treated is a cancer, for example, colon, colorectal, breast, brain, prostate, pancreatic or ovarian cancers, acute lymphocytic leukemia (ALL), acute myelogenous leukemia (AML), juvenile myelomonocytic leukemia (JMML), non-small cell lung cancer (NSCLC), small cell lung cancer (SCLC), lymphoma, melanoma, or myeloproliferative syndrome (MPS), osteosarcoma, neuroblastoma or glioblastoma. In further embodiments, the cancer is primary or metastatic. In yet further embodiments, the cancer is of the type represented by the cell line types shown in the Examples. In some embodiments, the subject having cancer possesses a mutation in the MEK and/or b-RAF, and/or KRAS gene(s) associated with an increased risk of cancer and/or resistance to certain anti-cancer treatments.

The embodiments described here are illustrative and are not meant to be limiting with regard to additional combination components, routes and order of administration, patient type (previously untreated or previously treated, absence or presence of co-morbid conditions, age, sex, etc.), or stage of patient's disease, type of a MEK inhibitor or a b-RAF inhibitor, or a KRAS inhibitor, etc.

The disclosed combinations and methods may afford one or more of the improvements as defined in the Glossary relative to the use of each individual components or existing single and combination treatments. Also, the disclosed combinations and methods may permit reduction in doses and/or frequency of administration of therapeutic agents and radiation to achieve the same improvements as a result of treatment relative to what is possible using individual components or existing single and combination treatments.

The disclosed combinations need not be synergistic or even result in a significant reduction in EC₅₀ values to yield a significant improvement in the effectiveness of treatment relative to single therapy with a MEK inhibitor or a b-RAF inhibitor, or a KRAS inhibitor. As discussed above, quiescent cancer cells are inherently less susceptible to anti-cancer therapeutics, including MEK inhibitors, b-RAF inhibitors, or KRAS inhibitors, and even a small fraction of quiescent cells that survives posttreatment can lead to recurrence. Consequently, eradicating the resistant, quiescent cell populations in a neoplasm may or may not yield a synergistic reduction in EC₅₀ values, yet may yield a significant improvement in cancer elimination, recurrence rate and appearance of metastatic neoplasms.

The administration routes and regimen of the disclosed combination may well vary depending on the neoplastic condition treated, extent of progression of the neoplasm, age and physical condition of the subject, exact combination selected, and other factors. Administration regimen may include multiple doses per period of time, the treatments administered concurrently or sequentially, etc. For example, DYRK1 inhibitor may be administered before a MEK inhibitor or a b-RAF inhibitor, or a KRAS inhibitor. The DYRK1 inhibitor may be administered 6 hours, 12 hours, 24 hours, 48 hours, 72 hours, 96 hours before a MEK inhibitor or a b-RAF inhibitor, or a KRAS inhibitor. The DYRK1 inhibitor may be administered at the same time (concomitantly) as a MEK inhibitor or a b-RAF inhibitor, or a KRAS inhibitor. The DYRK1 inhibitor may be administered 6 hours, 12 hours, 24 hours, 48 hours, 72 hours, 96 hours after a MEK inhibitor or a b-RAF inhibitor, or a KRAS inhibitor. The DYRK1 inhibitor and/or a MEK inhibitor or a b-RAF inhibitor, or a KRAS inhibitor (as described above), both or one of which may be administered before, after, or concomitantly with the radiation or other therapy.

The DYRK1 inhibitor may be administered at a different schedule regiment including but not limited to daily, every two days, every three days, every four days, biweekly (twice per week), once weekly, once every two weeks, once per month by oral (PO), intravenous (IV), intraperitoneal (IP), subcutaneous (SC), intratumoral (IT), intrathecal, or other routes of administration.

The combinations may be administered to subjects who are naive to treatment (have not been treated), or subjects who underwent previous treatments with first-line, second-line, third-line, or other therapies, radiation treatments, or have undergone surgical resection or de-bulking of a solid tumor, or subjects whose cancers relapsed, or subjects whose cancers are non-metastatic or metastatic.

### EXAMPLES

The following examples are not intended to be limiting. Those of skill in the art will, in light of the present disclosure, appreciate that many changes can be made in the specific materials and which are disclosed and still obtain a like or similar result without departing from the spirit and scope of the invention.

### Example 1. Determination of Fraction of Quiescent Cancer Cells within a Population

The following cell lines were obtained from ATCC and cultured according to the ATCC recommendations: H2122—non-small cell lung cancer cell line harboring G12C KRAS mutation; A549—non-small cell lung cancer cell line harboring G12S KRAS mutation; H23—non-small cell lung cancer cell line harboring G12C KRAS mutation; H358—non-small cell lung cancer cell line harboring G12C KRAS mutation; OVCAR—ovarian cancer cell line wild type KRAS, H1975—non-small cell lung cancer cell line wild-type KRAS, SW620—colon cancer cell line harboring G12V KRAS mutation. Cell cultures of these lines were seeded into 6-well plates at 3×10⁵-6×10⁵ cells/well; the plated number of cells depended on cell size and rate of proliferation, aiming for approximately 50% confluency. After seeding, the cells were allowed to attach for 24 hours while incubated at 37° C. in a humidified 5% CO₂ atmosphere, and then treated with compounds for desired amount of time (usually 24 hours) incubating under same conditions. Then the cells were harvested by trypsinization, pooled with the floating cells, washed in PBS, and fixed in 70% ice-cold ethanol overnight. For Acridine Orange (AO) staining, fixed cells were washed once with ice-cold PBS, re-suspended in 100 μL PBS, followed by addition of 200 μL of permeabilizing solution and 600 μL AO staining solution. The measurements were performed with Guava easyCyte HT flow cytometer (EMD Millipore) using the blue laser for excitation at 488 nm, monitoring emission of the AO-DNA complex at 526 nm and AO-RNA complex at 650 nm. The complete protocol and composition of buffers are described in the literature (Darzynkiewicz Z, Juan G, and Srour E F (2004) Differential Staining of DNA and RNA (2004). Current Protocols in Cytometry, Chapter 7:Unit 7.3).

### Example 2. General Procedure for the Cell Viability Assays in 2D Cell Culture

For viability analysis, cells were seeded into 96-well plates at 2×10³-6×10³ cells/well; depending on cell size and rate of proliferation aiming for approximately 50% confluency. Cells were allowed to attach for 24 hours incubated at 37° C. in a humidified 5% CO₂ atmosphere. The treatments were performed using at least 6 different concentrations of a compound in 1:3 serial dilutions. Before reading the results cells were incubated for 96 hours in 5% CO₂ incubator at 37° C. Each treatment was performed in triplicate. Results were analyzed by CellTiter-Glo™ Luminescent Cell Viability Assay (Promega, cat. #G7571) according to the manufacturer's instructions using SpectraMAX Gemini Spectrophotometer (Molecular Devices).

### Example 3. General Procedure for the Cell Viability Assays in 3D Cell Culture (Spheroids)

For viability analysis in 3D culture, cells were seeded into 96-well ULA (ultra-low attachment) plates (Corning #4515) at 5×10³-6×10³ cells/well depending on cell size and rate of proliferation aiming for spheroid formation with diameter of 400-600 μM at the beginning of treatment. Cells were incubated for 2-3 days (depending on the cell line) at 37° C. in a humidified 5% CO₂ atmosphere allowing for tight spheroid formation. For the treatment, 50 μL of media was removed from each well and replaced with fresh media with compounds. The treatments were performed using at least 6 different concentrations of a compound in 1:3 serial dilutions. Before reading the results, cells were incubated for the period of 4-10 days in 5% CO₂ incubator at 37° C. If the treatment time exceeded 4 days, 70 μL of media in each well was replaced with fresh media containing the test compound every fourth day. Each treatment was performed in duplicate. Results were analyzed by CellTiter-Glo™ 3D Luminescent Cell Viability Assay (Promega, cat. #G9682) according to the manufacturer's instructions using SpectraMAX Gemini Spectrophotometer (Molecular Devices). Prior to analysis, the spheroids were photographed at 50× magnification.

### Example 4. Combination of a DYRK1 Inhibitor with Selumetinib

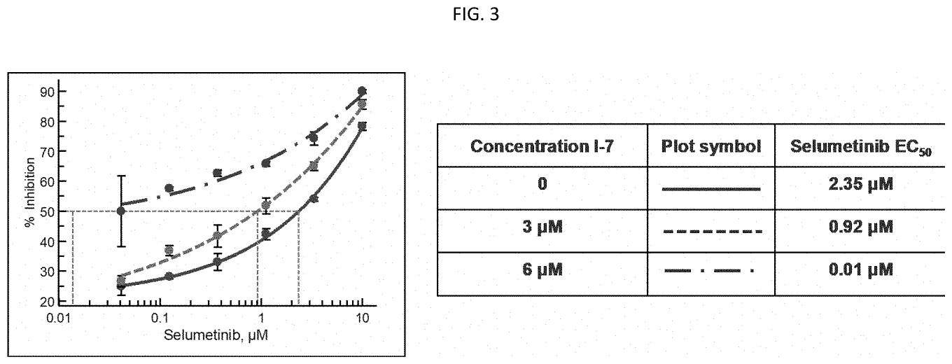

A549 cells were cultured and treated as described in Examples 1 and 2. The highest concentration of selumetinib used in this assay was 10 μM and the concentrations of Compound I-7 were 3 μM and 6 μM. The observed EC₅₀ values for selumetinib were 2.35 μM when Compound I-7 was not present, 0.92 μM when Compound I-7 was present at a concentration of 3 μM, and 0.01 μM when Compound I-7 was present at a concentration of 6 μM. See FIG. 3.

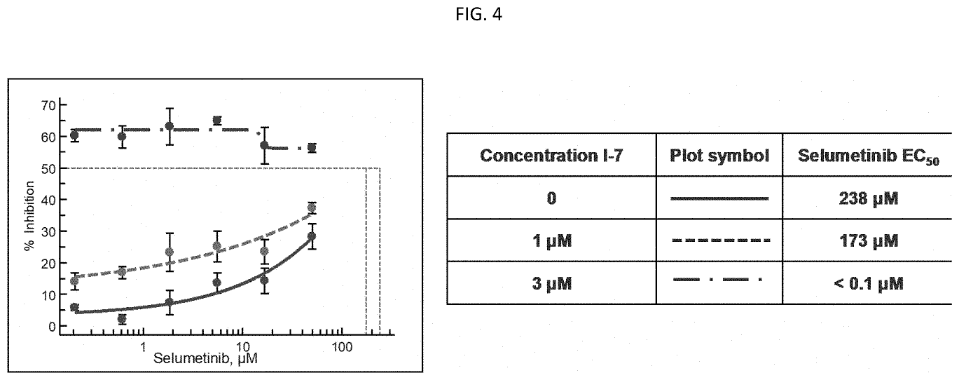

OVCAR cells were cultured and treated as described in Examples 1 and 2. The highest concentration of selumetinib used in this assay was 50 μM and the concentrations of Compound I-7 were 1 μM and 3 μM, respectively. The EC₅₀ values observed for selumetinib were 238 μM when Compound I-7 was not present, 173 μM when Compound I-7 was present at a concentration of 1 μM, and <0.1 μM when Compound I-7 was present at a concentration of 3 M. See FIG. 4.

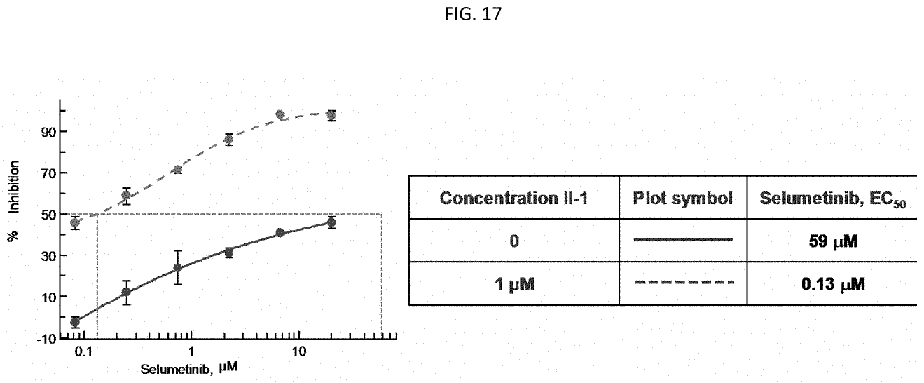

H1975 cells were cultured and treated as described in Examples 1 and 2. The highest concentration of selumetinib used in this assay was 20 μM and the concentrations of Compound II-1 were 1 μM. The EC₅₀ values observed for selumetinib were 59 μM when Compound II-1 was not present, 0.13 μM when Compound II-1 was present at a concentration of 1 μM. See FIG. 17.

In these experiments it was demonstrated that combining a reversible MEK inhibitor and a b-RAF inhibitor selumetinib with DYRK1B inhibitor, Compound I-7, demonstrated a significant increase in cytotoxicity (decrease in ECso value) of selumetinib against A549 non-small cell lung cancer cell line harboring KRAS G12S mutation. Further, a synergistic effect, i.e. significant increase in cytotoxicity (lower ECso value) was observed against H1975 non-small cell lung cancer cell line and OVCAR ovarian cancer cell line both are wild type KRAS when treated with a combination of DYRK1B inhibitor, Compound II-1 and a reversible MEK inhibitor and a b-RAF inhibitor selumetinib.

### Example 5. Combination of a DYRK1 Inhibitor with Trametinib

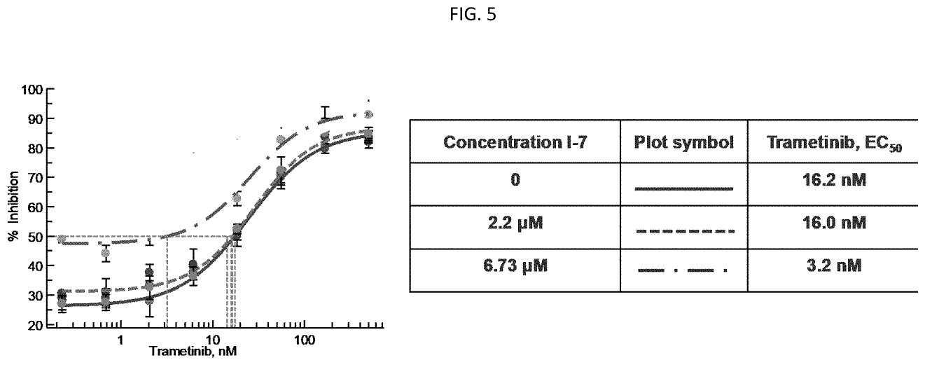

A549 cells were cultured and treated as described in Examples 1 and 2. The highest concentration of trametinib used in this assay was 0.5 μM and the concentrations of Compound I-7 were 2.2 μM and 6.73 μM, respectively. The ECso values observed for trametinib were 16.2 nM when Compound I-7 was not present, 16 nM when Compound I-7 was present at a concentration of 2.2 μM, and 3.2 nM when Compound I-7 was present at a concentration of 6.73 μM. See FIG. 5.

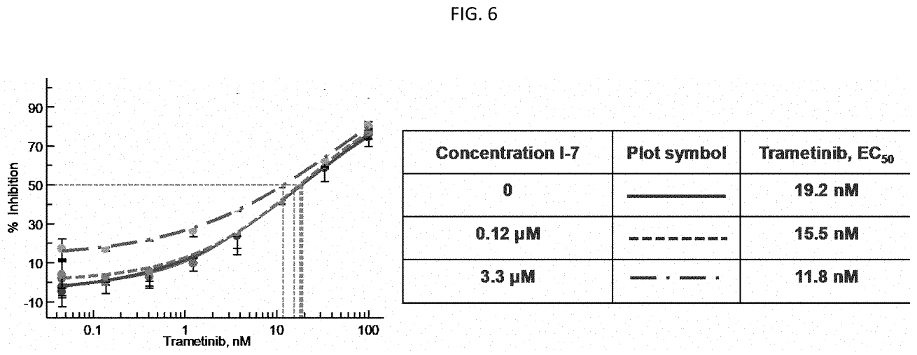

H23 cells were cultured and treated as described in Examples 1 and 2. The highest concentration of trametinib used in this assay was 100 nM and the concentrations of Compound I-7 were 0.12 μM and 3.3 μM, respectively. The ECso values observed for trametinib were 19.2 nM when Compound I-7 was not present, 15.5 nM when Compound I-7 was present at a concentration of 0.12 μM, and 11.8 nM when Compound I-7 was present at a concentration of 3.3 μM. See FIG. 6.

### Example 6. Combination of a DYRK1 Inhibitor with Trametinib in 3D Cell Culture

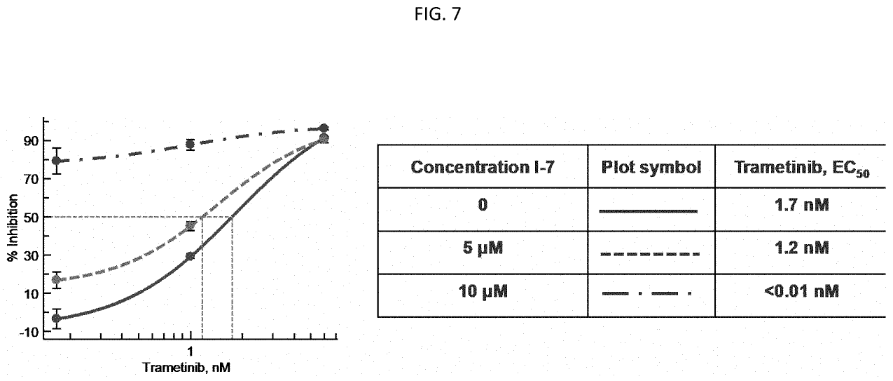

H23 cells were cultured and treated as described in Examples 1 and 5. The highest concentration of trametinib used in this assay was 6 nM and the concentrations of Compound I-7 were 5 μM and 10 μM, respectively. The EC₅₀ values observed for trametinib were 1.7 nM when Compound I-7 was not present, 1.2 nM when Compound I-7 was present at a concentration of 5 μM, and <0.01 nM when Compound I-7 was present at a concentration of 10 μM. See FIG. 7.

In this experiment it was demonstrated that combining an irreversible MEK inhibitor trametinib with Compound I-7 yielded a significant increase in cytotoxicity (lower EC₅₀ value) of trametinib against A549 cells and H23 non-small cell lung cancer cells. Such combinations are synergistic and may well yield a significant improvement in the effectiveness of treatment relative to single therapy with a MEK inhibitor by eradicating the resistant, quiescent cell populations in a neoplasm that otherwise survive the single therapy treatment.

### Example 7. Combination of a DYRK1 Inhibitor with KRAS G12C Mutant Inhibitor MRTX-849 or KRAS G12C Mutant Inhibitor AMG-510

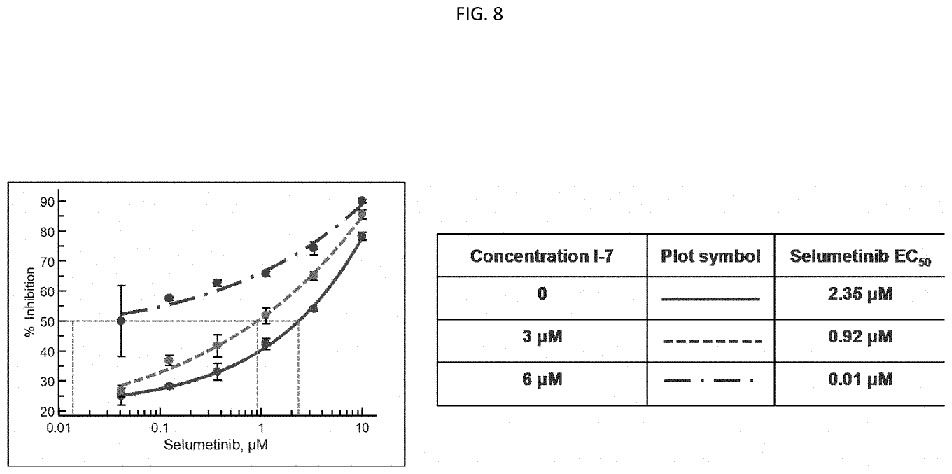

H2122 cells were cultured and treated as described in Examples 1 and 2. The highest concentration of MRTX-849 used in this assay was 1 μM and the concentrations of Compound I-7 were 2.5 μM and 5 μM. The EC₅₀ values observed were 0.4 μM when Compound I-7 was not present, 0.1 μM when Compound I-7 was present at a concentration of 2.5 μM, and 0.018 μM when Compound I-7 was present at a concentration of 5 μM. See FIG. 8.

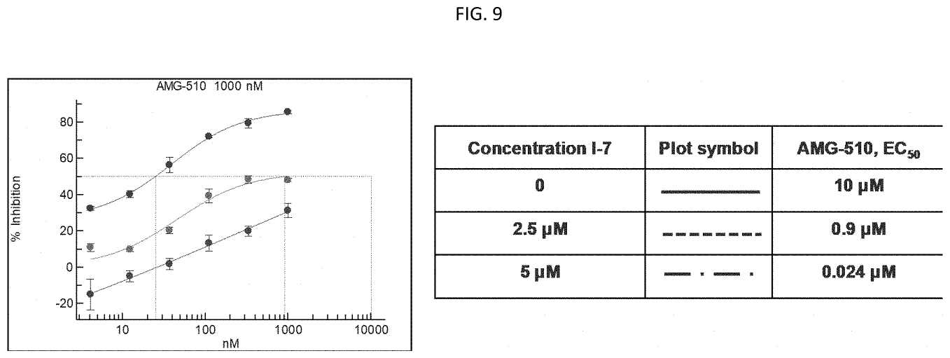

H2122 cells were cultured and treated as described in Examples 1 and 2. The highest concentration of AMG-510 used in this assay was 1 μM and the concentrations of Compound I-7 were 2.5 μM and 5 μM. The EC₅₀ values observed were >1 μM when Compound I-7 was not present, 0.9 μM when Compound I-7 was present at a concentration of 2.5 μM, and 0.024 μM when Compound I-7 was present at a concentration of 5 μM. See FIG. 9.

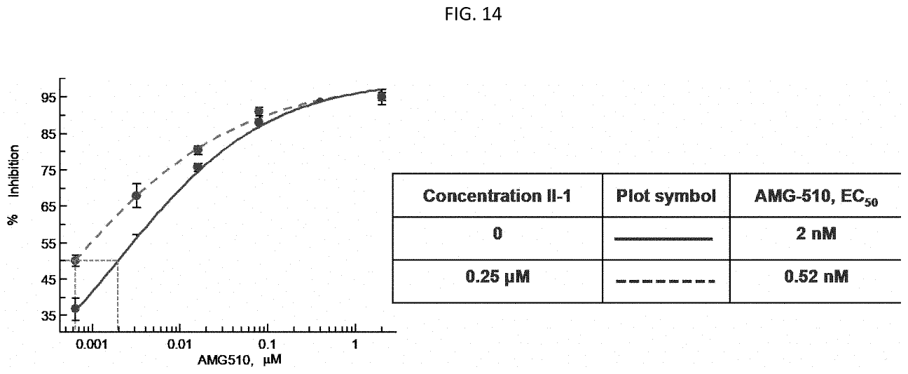

H358 cells were cultured and treated as described in Examples 1 and 2. The highest concentration of AMG-510 used in this assay was 2 μM and the concentrations of Compound II-1 was 0.25 μM. The EC₅₀ values observed were 2 nM when Compound II-1 was not present, 0.53 nM when Compound II-1 was present at a concentration of 0.25 μM. See FIG. 14.

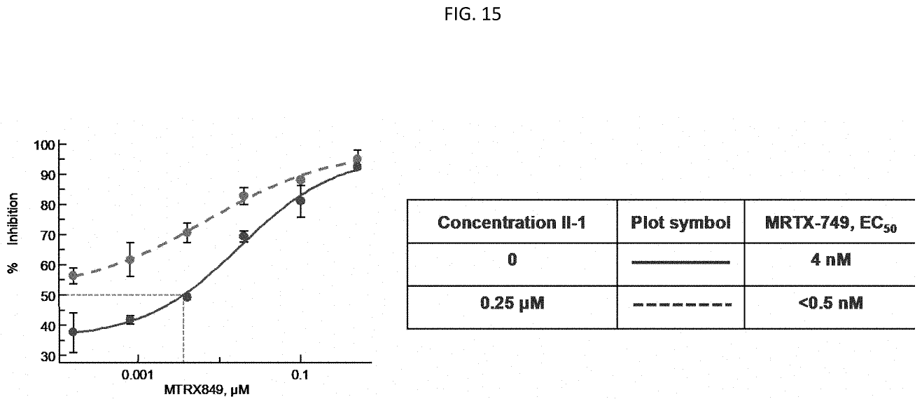

H358 cells were cultured and treated as described in Examples 1 and 2. The highest concentration of MRTX-849 used in this assay was 0.2 μM and the concentration of Compound II-1 were 0.25 μM. The EC₅₀ values observed were 4 nM when Compound II-1 was not present, <0.5 nM when Compound II-1 was present at a concentration of 0.25 μM. See FIG. 15.

In these experiments it was demonstrated that combining a KRAS G12C mutant inhibitor MRTX-849 or AMG-510 with Compound I-7 yielded a significant increase in cytotoxicity (lower EC₅₀ value) of either MRTX-849 or AMG-510 against H2122 cells. Further, it was demonstrated that combining either MRTX-849 or AMG-510 with Compound II-1 yielded a significant increase in cytotoxicity against H358 non-small cancer cells. Those combinations were synergistic and result in a dramatic reduction in EC₅₀ values, it may well yield a significant improvement in the effectiveness of treatment relative to single therapy with a KRAS G12C mutant inhibitor by eradicating the resistant and quiescent cell populations in a neoplasm that otherwise survive the single therapy treatment.

### Example 8. Cell Cycle Effects and Cytotoxicity of Trametinib and Combination of a DYRK1 Inhibitor and Trametinib

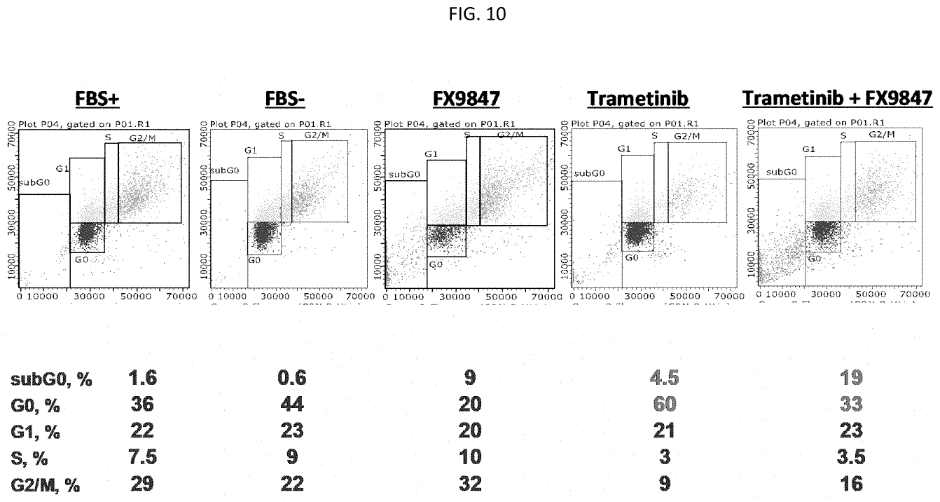

SW620 cells were cultured, treated, and analyzed as described in Example 1. The results when different concentrations of trametinib, Compound I-7, or both trametinib and Compound I-7 are present are shown in FIG. 10.

In these experiments it was demonstrated that exposure of SW620 cells to trametinib results in changes in cell cycle distribution, such that trametinib is even more effective than serum starvation (FBS−) at inducing a large fraction of the cells into the quiescent (G₀) state. The cell cycle distribution of normally proliferating SW620 cells, those incubated in serum free media (FBS−) and regular growth medium (FBS+), is shown for comparison. The trametinib caused changes to the cell cycle distribution whether the cells were pre-incubated in normal growth medium (FBS+) or pre-starved in serum free (FBS−) medium to increase proportion of cells in G₀ prior to treatment with trametinib. Further, combination of Compound I-7 with trametinib reduced the fraction of cells in G₀ and strongly enhanced the resultant cytotoxicity as recorded by significant increases in apoptotic cells demonstrated as sub-G₀ population and demonstrated by viability assays. This effect was observed irrespective of whether or not the cells were pre-incubated in growth medium or under the conditions of serum starvation.

### Example 9. Cell Cycle Effects and Cytotoxicity of MRTX-849 and Combination of DYRK1 Inhibitor with MRTX-849

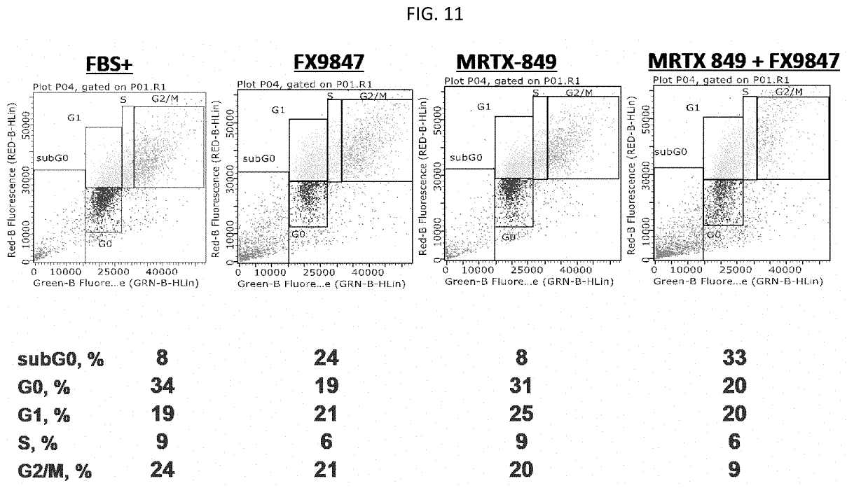

The H2122 cells were cultured and treated as described in Example 1. The results when cells were treated with MRTX-849, Compound I-7, or both MRTX-849 and Compound I-7 are present are shown in FIG. 11.

In these experiments it was demonstrated that exposure of H2122 cells to MRTX-849 results in changes in cell cycle distribution, such that MRTX-849 does not allow cells to exit the quiescent (G₀) state therefore these cells survive the treatment. The MRTX-849 caused changes to the cell cycle distribution when the cells were pre-incubated in normal growth medium (FBS+) Further, combination of Compound I-7 with MRTX-849 reduced the fraction of cells in G₀ and strongly enhanced the resultant cytotoxicity as recorded by significant increases in apoptotic cells demonstrated as sub-G₀ population and demonstrated by viability assays.

### Example 10. Cell Cycle Effects and Cytotoxicity of AMG-510 and Combination of DYRK1 Inhibitor with AMG-510

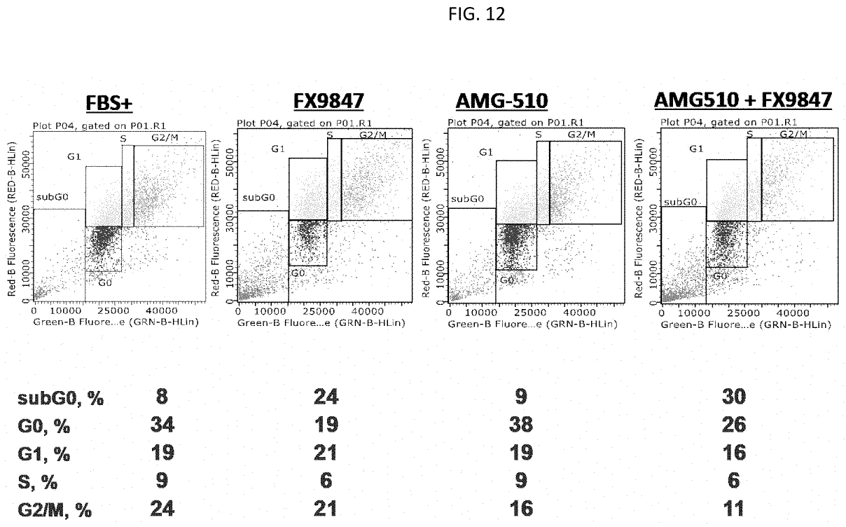

The H2122 cells were cultured, treated, and analyzed as described in Examples 1 and 2. The results when cells were treated with AMG-510, Compound I-7, or both AMG-510 and Compound I-7 are present are shown in FIG. 12.

In this example, H2122 cells were incubated for 24 hours under normal growth medium (FBS+) with or without treatment. Under these conditions, exposure to AMG-510 led to an increase in fraction of cells in quiescent state (G₀). When cells were co-treated with combination of AMG-510 and Compound I-7 no such increase in proportion of quiescent cells was observed and a large increase in cytotoxicity of AMG-510 was observed, as judged by the large increase in apoptotic cells as determined by sub-G₀ fraction.

### Example 11. Induction of DYRK1B Upon Treatment of SW620 Cells with Trametinib, MEK Inhibitor

SW620 cells were cultured and treated as described in Examples 1 and 2. For Western Blot analysis cells were seeded into 6-well plates at 5×10⁵-9×10⁵ cells/well (depending on the cell size and rate of proliferation), allowed to attach for 24 hours, then treated with compounds for 24 hours, and harvested. Immunoblotting was performed using conventional techniques, as described in Cell Signaling Technologies Western Blotting protocol (www.cellsignal.com).

Antibodies used for blotting were from Cell Signaling Technology (CST): DYRK1B (D40D1) Rabbit mAb #5672; β-Actin (13E5) Rabbit mAb #4970; Anti-rabbit IgG, HIRP-linked Antibody #7074. The Primary Antibody Dilution Buffer 1×TBST with 5% BSA (CST #9998) was used. For detection, SignalFire™ ECL Reagent (CST #6883) was used.

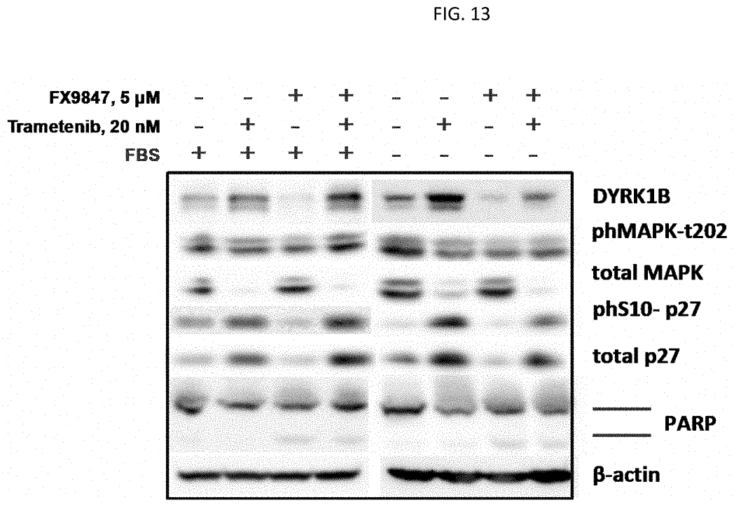

The expression levels of DYRK1B, ph-t202 MAPK, total MAPK, ph-S10 p27, total p27 and β-actin in SW620 cells following the 24 hours treatment with trametinib or combination of trametinib and compound I-7 as observed by Western blot analysis are shown in FIG. 13. The expression of DYRK1B protein was compared to that in untreated cells incubated in regular growth medium containing FBS (FBS+) or serum free medium (FBS−), single treatment with trametinib to the treatment with combination of trametinib and compound I-7. It was demonstrated that treatment with trametinib suppressed the MAPK expression, induced the phosphorylation of p27, induced the expression of p27, and also induced expression of DYRK1B protein even higher than that produced by serum starvation. The combination treatment with trametinib and compound I-7 demonstrated a decrease of the phosphorylation of p27 and the total levels of p27 as compared to the treatment with trametinib alone.

### Example 12. General Procedure for the Cell Viability Assays in 3D Cell Culture (Spheroids)

For viability analysis in 3D culture, cells were seeded into 96-well ULA (ultra-low attachment) plates (Corning #4515) at 5×10³-6×10³ cells/well depending on cell size and rate of proliferation aiming for spheroid formation with diameter of 400-600 μM at the beginning of treatment. Cells were incubated for 2-3 days (depending on the cell line) at 37° C. in a humidified 5% CO₂ atmosphere allowing for tight spheroid formation. For the treatment, 50 μL of media was removed from each well and replaced with fresh media with compounds. The treatments were performed using at least 6 different concentrations of a compound in 1:3 serial dilutions. Before reading the results, cells were incubated for the period of 4-10 days in 5% CO₂ incubator at 37° C. If the treatment time exceeded 4 days, 70 μL of media in each well was replaced with fresh media containing the test compound every fourth day. Each treatment was performed in duplicate. Results were analyzed by CellTiter-Glo™ 3D Luminescent Cell Viability Assay (Promega, cat. #G9682) according to the manufacturer's instructions using SpectraMAX Gemini Spectrophotometer (Molecular Devices). Prior to analysis, the spheroids were photographed at 50× magnification

While this invention has been particularly shown and described with references to example embodiments thereof, it will be understood by those skilled in the art that various changes in form and details may be made therein without departing from the scope of the invention encompassed by the appended claims.

1. A method for treating a subject having a neoplasm, the method comprising administering to the subject, sequentially or concomitantly,
(a) a first agent being a DYRK1 inhibitor which inhibits DYRK1A or DYRK1B kinase activity with an IC₅₀ of 100 nM or lower in a radiolabeled kinase biochemical assay, and reduces the fraction of quiescent cancer cells representative of the neoplasm being treated (in vitro or in vivo) that would otherwise be found in the absence of such inhibitor by at least 10%, as quantitated by FACS;
(b) administering to the subject a second agent being an inhibitor of the MAPK pathway; thereby treating the subject having the neoplasm.

2. The method of claim 1, wherein EC₅₀ of the second agent of is reduced by at least 2-fold in cell-based assays.

3. The method of claim 1, further comprising administering to the subject an effective amount of radiation therapy.

4. The method of claim 1, wherein the neoplasm being treated is either a primary or a metastatic cancer selected from biliary cancer, brain cancer, breast cancer, cervical cancer, colon cancer, gastric cancer, kidney cancer, head and neck cancer, leukemia, liver cancer, lung cancer, small cell lung carcinoma, non-small lung SCC, lymphoma, ovarian cancer, pancreatic cancer, prostate cancer, rectal cancer, sarcoma, skin cancer, testicular cancer, thyroid cancer, uterine cancer, bladder cancer, breast cancer, colorectal cancer, ovarian cancer, and prostate cancer.

5. The method of claim 1, wherein the MAPK pathway inhibitor is a MEK inhibitor, either of MEK1 or of MEK2.

6. The method of claim 1, wherein a MEK inhibitor is selected from trametinib, cobimetinib, binimetinib, selumetinib, PD-325901, or CI-1040.

7. The method of claim 1, wherein the MAPK pathway inhibitor is an inhibitor of wild-type or mutated (mutant) KRAS, including but not limited to KRAS mutants on codons 12, 13, and 61, such as G12C, G12D, G12V, G13D, and Q61H.

8. The method of claim 1, wherein the KRAS-mutant inhibitor is a pan-RAS inhibitor is selected from BI 1701963, an inhibitor of certain mutant KRAS proteins, such as Sotorasib (AMG 510) and MRTX849.

9. The method of claim 1, the MAPK pathway inhibitor is a MEK inhibitor, either MEK1 or MEK2. In a further embodiment, a MAPK pathway inhibitor is an inhibitor of wild-type or mutated (mutant) KRAS, including but not limited to KRAS mutants on codons 12, 13, and 61, such as G12C, G12D, G12V, G13D, and Q61H.

10. The method of claim 1, The method of claim 1, wherein the DYRK1 inhibitor is selected from I-1, I-2, I-3, I-4, I-5, I-6, and I-7.

11. A method of claim 1, wherein the EC₅₀ value of the inhibitor of MAPK kinase pathway is at least 20% lower in the combination treatment when compared to the same treatment with the inhibitor of the MARK kinase pathway alone, as determined in cells representative of the neoplasm being treated by a luminescent cell viability assay.

12. The method of claim 1, wherein the DYRK1 inhibitor of Formula I: 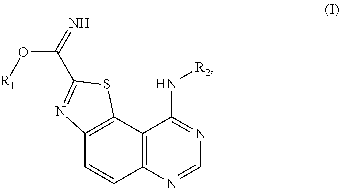
or a pharmaceutically acceptable salt or solvate thereof, wherein
R₁ is a substituted or unsubstituted C₁₋₈ alkyl, a substituted or unsubstituted phenyl, or a substituted or unsubstituted benzyl;
R₂ is phenyl, optionally substituted with up to four groups independently selected from halo, CN, NO₂, NHC(O)C₁₋₄ alkyl, C₁₋₄ alkyl, OH, OC₁₋₄ alkyl, wherein two adjacent groups and their intervening carbon atoms may form a 5- to 6-membered ring containing one or more heteroatoms selected from N, O, or S.

13. The method of claim 12, wherein the DYRK1 inhibitor selected from Formulas I-1, I-2, I-3, I-4, I-5, I-6, and I-7.

14. The method of claim 1, wherein the DYRK1 inhibitor of Formula II: 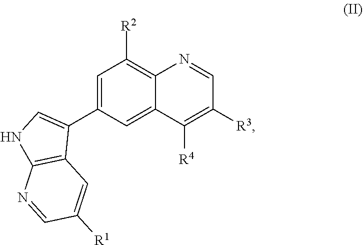
or a salt, stereoisomer, tautomer or N-oxide thereof,
wherein
R¹, R³, R⁴ are independently selected from the group consisting of
(vii) H, halogen, CN, NO₂, C₁-C₆-alkyl, C₂-C₆-alkenyl, C₂-C₆-alkynyl; wherein each substitutable carbon atom in the aforementioned moieties is independently unsubstituted or substituted with one or more, same or different substituents R⁷;
(viii) C(═O)R⁵, C(═O)OR⁶, C(═O)SR⁶, C(═O)N(R^{6a})(R^{6b}), OR⁶, S(═O)ₙR⁶, S(═O)ₙN(R^{6a})(R^{6b}), S(═O)ₙOR⁶, N(R^{6a})(R^{6b}), N(R⁶)C(═O)R⁵, N(R⁶)C(═O)OR⁶, N(R⁶)C(═O)N(R^{6a})(R^{6b}), N(R⁶)S(═O)ₙR⁶, N(R⁶)S(═O)ₙN(R^{6a})(R^{6b}), N(R⁶)S(═O)ₙOR⁶;
(ix) a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R⁸;
R² is selected from the group consisting of H, halogen, CN, NO₂, C₁-C₆-alkyl, C₁-C₆-haloalkyl, C₂-C₆-alkenyl, C₂-C₆-alkynyl, C₁-C₆-alkoxy and C₁-C₆-haloalkoxy;
R⁵, R⁶, R^{6a}, R^{6b} are independently selected from the group consisting of H, C₁-C₆-alkyl, C₁-C₆-haloalkyl, C₂-C₆-alkenyl, C₂-C₆-alkynyl, C₁-C₆-alkylcarbonyl, wherein each substitutable carbon atom in the aforementioned moieties is independently unsubstituted or substituted with one or more, same or different substituents R⁹; and a 3- to 9-membered saturated, partially unsaturated or fully unsaturated carbocyclic or heterocyclic ring, wherein said heterocyclic ring comprises one or more, same or different heteroatoms selected from O, N or S, wherein said N and/or S-atoms are independently oxidized or non-oxidized, and wherein each substitutable carbon or heteroatom in the aforementioned cyclic moieties is independently unsubstituted or substituted with one or more, same or different substituents R¹⁰;
R⁷ is selected from the group consisting of halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(═O)R⁵, C(═O)OR⁶, C(═O)SR⁶, C(═O)N(R^{6a})(R^{6b}), OR⁶, S(═O)ₙR⁶, S(═O)ₙN(R^{6a})(R^{6b}), S(═O)ₙOR⁶, N(R^{6a})(R^{6b}), N(R⁶)C(═O)R⁵, N(R⁶)C(═O)OR⁶, N(R⁶)C(═O)N(R^{6a})(R^{6b}), N(R⁶)S(═O)ₙ(R⁶), N(R⁶)S(═O)ₙN(R^{6a})(R^{6b}), N(R⁶)S(═O)ₙOR⁶; and a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned cyclic or bicyclic moieties is unsubstituted or substituted with one or more, same or different substituents R⁸;
R⁸ is selected from the group consisting of halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkylcarbonyl, N(R^{6a})(R^{6b}), OR⁶ and S(═O)ₙR⁶;
R⁹ is selected from the group consisting of halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkylcarbonyl, N(R^{11a})(R^{11b}), OR¹¹ and S(═O)ₙR¹¹; and a 3- to 9-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned cyclic moieties is unsubstituted or substituted with one or more, same or different substituents R¹⁰;
R¹⁰ is selected from halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkylcarbonyl, N(R^{11a})(R^{11b}), OR¹¹ and S(═O)ₙR¹¹;
R¹¹, R^{11a}, R^{11b} are independently selected from the group consisting of H, C₁-C₆-alkyl, C₂-C₆-alkenyl and C₂-C₆-alkynyl;
and wherein
n is 0, 1 or 2.
In one embodiment, R¹ is selected from the group consisting of
(vii) H, halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl;
wherein each substitutable carbon atom in the aforementioned moieties is independently unsubstituted or substituted with one or more, same or different substituents R⁷;
(viii) C(═O)R⁵, C(═O)OR⁶, C(═O)SR⁶, C(═O)N(R^{6a})(R^{6b}), OR⁶, S(═O)ₙR⁶, S(═O)ₙN(R^{6a})(R^{6b}), S(═O)ₙOR⁶, N(R^{6a})(R^{6b}), N(R⁶)C(═O)R⁵, N(R⁶)C(═O)OR⁶, N(R⁶)C(═O)N(R^{6a})(R^{6b}), N(R⁶)S(═O)ₙR⁶, N(R⁶)S(═O)ₙN(R^{6a})(R^{6b}), N(R⁶)S(═O)ₙOR⁶;
preferably R¹ is selected from the group consisting of H, halogen, CN, NO₂, C₁-C₃-alkyl, C₂-C₃-alkenyl, C₂-C₃-alkynyl and C(═O)N(R^{6a})(R^{6b});
wherein each substitutable carbon atom in the aforementioned moieties is independently unsubstituted or substituted with one or more, same or different substituents R⁷;
more preferably R¹ is selected from the group consisting of H, halogen, CN, NO₂, C₁-C₃-alkyl, C₂-C₃-alkenyl and C₂-C₃-alkynyl;
wherein each substitutable carbon atom in the aforementioned moieties is independently unsubstituted or substituted with one or more, same or different substituents R⁷.

15. The method of claim 1, wherein the combination treatment increases the fraction of apoptotic cells in a treated population of cells representative of the neoplasm being treated as compared to either agent alone by at least by 2-fold, as determined by the fraction of sub-G₀ cells by a FACS assay.

16. The method of claim 1, wherein the subject has been treated with one or more inhibitors of MAPK pathway and has acquired resistance to the same.

17. The method of claim 1, wherein the subject is human.
